(19) United States
(12) Patent Application Publication
(10) Pub. No.: US 2024/0257959 A1
(43) Pub. Date: Aug. 1, 2024

(54) ALIGNING TIMESTAMPS OF DATA COLLECTED BY MULTIPLE DEVICES AT MEDICAL SCENE

(71) Applicant: ZOLL Medical Corporation, Chelmsford, MA (US)

(72) Inventors: James P. O’CONNOR, Billerica, MA (US); Brett BONNER, Littleton, MA (US)

(73) Assignee: ZOLL Medical Corporation, Chelmsford, MA (US)

(21) Appl. No.: 18/422,826

(22) Filed: Jan. 25, 2024

Related U.S. Application Data
(60) Provisional application No. 63/441,358, filed on Jan. 26, 2023.

(51) Int. Cl. G16H 40/40; G16H 40/67

(52) U.S. Cl. CPC G16H 40/40; G16H 40/67

(57) ABSTRACT

In an illustrative embodiment, systems and methods for aligning data timing of data sets collected by devices used at a medical scene may include a first device configured to establish wireless communication with at least a portion of the devices, and repeatedly, during treatment of a patient at the medical scene, receive a message including a current device time as determined by each device. The first device may determine a receipt time of the message and store a timing correlation based on the current device time, the receipt time, and a device identifier. Each timing correlation may be applied as an offset to timestamps of a corresponding data set, each data set collected by a respective device of the devices, to align timing of the data sets, thereby enabling combined analysis across the data sets.

### RELATED APPLICATIONS

The present application claims priority to U.S. Provisional Application No. 63/441,358 entitled “Aligning Timestamps of Data Collected by Multiple Devices at Medical Scene” and filed Jan. 26, 2023. All above identified applications are hereby incorporated by reference in their entireties.

### BACKGROUND

Multiple medical devices may be used in medical situations (for example, emergency situations). These devices can be used by different personnel. For example, automated external defibrillators (AEDs) may be used by non-trained medical device personnel such as first responders. Additionally, emergency medical technicians (EMTs) may use different or additional devices in responding to an emergency situation, which may differ from devices used at a hospital. In addition, there may be one or more information display devices such as liquid-crystal display (LCD) panels, portable computing devices such as tablets, mobile communication devices (e.g., iPhone), smart watches (e.g., iPad, Apple Watch provided by Apple, Inc.), or other types of wearable computing and display devices, upon which information from the one or more medical devices can be presented.

In one example, the medical situation is sudden cardiac arrest, which is a frequent cause of death. One treatment for cardiac arrest is quick and competent chest compressions to keep blood flowing from a patient's heart to vital parts of the body. Along with chest compressions, a rescuer can ventilate the patient by either providing positive pressure breaths into the patient's mouth or nose or utilizing a device that pushes air into the patient's lungs. Rescuers, such as lay responders, emergency medical technicians (EMTs), rescue team supervisors, paramedics, doctors, or other rescuers, can benefit from feedback about performance of cardiopulmonary resuscitation (CPR) and from information about the patient's medical status during treatment of the patient. Information about the patient's health status, physiologic data and information about treatment delivered to the patient can be collected by sensors.

### SUMMARY OF ILLUSTRATIVE EMBODIMENTS

In one aspect, the present disclosure relates to a system for aligning data timing of a number of data sets collected by a number of devices used at a medical scene, the system including a first device of the number of devices including a non-volatile data store, at least one wireless communications interface configured for bi-directional communications using a wireless communication protocol, and processing circuitry. The processing circuitry may be configured to establish, using the at least one wireless communications interface, wireless communication with at least a portion of the number of devices, repeatedly, for each respective device of the at least the portion of the number of devices during treatment of a patient at the medical scene, receive, via the at least one wireless communications interface, a message including a current device time as determined by the respective device, determine a receipt time of receipt of the message, and store, to the non-volatile data store, a timing correlation based on the current device time, the receipt time, and a device identifier of the respective device. Each respective timing correlation for the at least the portion of the number of devices may be configured to be applied as an offset to timestamps of a corresponding data set of the number of data sets, each data set collected by a respective device of the number of devices, to align timing of the number of data sets, thereby enabling combined analysis across at least a portion of the number of data sets.

In some embodiments, the wireless communication protocol is Bluetooth, Wi-Fi, or Zigbee protocol. Establishing the wireless communication may include broadcasting a timing request to at least a portion of the number of devices. Establishing the wireless communications may include broadcasting a data request to at least a portion of the number of devices. Establishing the wireless communication may include establishing secure wireless communication using at least one of encryption or a proprietary communication protocol.

In some embodiments, the current device time is provided at least to a second accuracy. The current device time may be provided at least to a tenth of a second accuracy. For one or more devices of the number of devices, the message including the current device time may include at least one data value collected from the respective device. At least one device of the one or more devices may be configured to transmit, in real time, the current device time and at least one data value collected from the respective device. The at least one device may include a sensor device, and the at least one data value is a sensed value.

In some embodiments, the processing circuitry is further configured to periodically issue a request to the at least the portion of the number of devices, and receiving the message includes, for one or more devices of the at least the portion of the number of devices, receiving a response from the request. Periodically issuing the request may include broadcasting the request wirelessly to all devices within range. Periodically issuing the request may include issuing the request once every one to five minutes.

In some embodiments, at least one device of the at least the portion of the plurality devices is configured to transmit, in real time, a data stream including a number of data values, and the processing circuitry is further configured to store, to the non-volatile data store, a data log for the at least one device. Storing the timing correlation may include storing, in the data log, the timing correlation. Each device of the at least one device may be a medical device or a sensor device, and the first device may be a patient monitoring device. The data stream may include data collected by two or more devices connected to the at least one device as two or more peripheral devices.

In some embodiments, at least one device is a medical device or a patient monitoring device. The at least the portion of the number of devices may include the two or more peripheral devices. The at least one device may include two or more devices, and the processing circuitry may be further configured to, for each respective device of the at least one device, apply an offset derived from the respective timing correlation to at least a portion of a number of timestamps of a data log of the respective device.

In some embodiments, two or more devices of the at least the portion of the plurality devices are configured to transmit, in real time, a respective data stream including a number of data values, and the processing circuitry is further configured to apply, in real-time, timing correlations corresponding to each respective device of the two or more devices to the respective data stream of the respective device to synchronize the data values of the data streams of the two or more devices. The processing circuitry may be further configured to present, at the display in real-time or near real-time, co-registered graphical outputs of the number of data values corresponding to the data streams of at least two devices of the two or more devices. The first device may include the display.

In some embodiments, the processing circuitry is further configured to present, at the display in real-time or near real-time, one or more time-based metrics combining synchronized data values of the data streams of at least two devices of the two or more devices. The first device may include the display.

In some embodiments, the first device is a medical device configured to deliver a therapy to a patient. The first device may be a patient monitoring device or a portable computing device executing a clinical application. The first device may be an edge server.

In some embodiments, storing the timing correlation includes storing the current device time, the receipt time, and the device identifier to a timing marker data file including correlations for each device of the at least the portion of the number of devices. The processing circuitry may be further configured to provide, via a network, the timing marker data file to another computing device. The number of devices may include the another computing device, and the processing circuitry may be configured to negotiate, with the another computing device, for role as master device updating the timing marker data file.

In some embodiments, the processing circuitry is configured to upload a set of timing correlations corresponding to the portion of the number of devices via a network to a cloud-based data analytics platform. The cloud-based data analytics platform may include processing logic configured to receive, separately, at least a portion of the number of data sets collected by the number of devices, and apply the set of timing correlations to the portion of the number of data sets corresponding to the at least the portion of the number of devices to synchronize the data values of the portion of the number of data sets. The system may include the cloud-based analytics platform.

In some embodiments, the first device includes an audio input, and the processing circuitry is further configured to record, via the audio input, a statement uttered by a caregiver at the medical scene, apply at least one natural language processing algorithm to the statement to identify a verbal marker indicating one of a beginning of a therapy, an end of the therapy, a delivery of a pharmaceutical treatment, or a performance of a treatment, and, responsive to identifying the verbal marker, add the verbal marker and a timestamp corresponding to the verbal marker as another timing correlation. The another timing correlation may be configured to be applied as an offset to timestamps of a given data set of the number of data sets representing data related to the therapy or the performance of the treatment.

In some embodiments, the present disclosure relates to a system for aligning data timing of a number of data sets collected by a number of devices used at a medical scene, the system including a master device of the number of devices, the master device including a non-volatile memory, at least one wireless communications interface, and device processing circuitry. The device processing circuitry may be configured to repeatedly, during treatment of a patient at the medical scene, determine a current master time of a series of master times of the respective device, and issue, via the at least one wireless communications interface for receipt by other devices of the number of devices, a timing request. The device processing circuitry may be configured to correlate, in the non-volatile memory, the current master time with at least one of i) issuance of the timing request, ii) a timestamp of a response from each device of at least a portion of the other devices, or iii) data provided by each device of at least a portion of the other devices. The system may include an analytics platform including a non-volatile data archive, at least one network interface, and platform processing circuitry configured to collect, from each device of the number of devices, a respective data set of the number of data sets, identify, using the series of master times, at least one time difference between the master device and each device of the one or more other devices, and apply the at least one time difference to align each data set of the number of data sets with the series of master times.

In some embodiments, the analytics platform is an edge server. The analytics platform may include a number of cloud-based resources. The collecting, the identifying, and the applying may be performed repeatedly in real-time or near real-time during the treatment of the patient at the medical scene.

In some embodiments, issuing the timing request includes issuing a uni-directional broadcast message. Issuing the timing request may include issuing a bi-directional request directed to each device of at least a portion of the other devices. Correlating the current master time may include correlating the current master time with the timestamp of each response of a number of responses received from at least a portion of the other devices in a master timing log. Correlating the current master time may include correlating, for each respective device of at least a portion of the one or more other devices, the current master time with a data entry or a beginning of a series of data entries provided by the respective device.

In some embodiments, collecting the number of data sets includes receiving upload of at least a portion of the number of data sets via a network connection. Identifying the at least one time difference may include, for each respective device of the other devices, calculating a respective time difference of one or more time differences corresponding to one or more times of the series of master times relevant to the respective device. Identifying the at least one time difference may include, for each respective device of at least a portion of the other devices, identifying, in the data set of the respective device, at least one fiducial marker each representing a respective timing difference between the respective device and the master device at a time of entry of the fiducial marker.

In some embodiments, the at least one fiducial marker includes the current master time as issued in the corresponding timing request. Each respective fiducial marker of the at least one fiducial marker may include a respective message identifier of the corresponding timing request, where each time entry of the series of master times includes a corresponding message identifier. Each respective fiducial marker of the at least one fiducial marker may include a device time of the respective device.

In some embodiments, applying the at least one time difference includes, for each respective data set of at least a portion of the number of data sets, adjusting a series of timestamps of the respective data set.

In one aspect, the present disclosure relates to a system for aligning data timing of a number of data sets collected by a number of devices used at a medical scene, the system including a non-volatile data archive, and processing circuitry configured to access, from the non-volatile data archive, a respective data set of a number of data sets. The number of data sets may represent data collected from the number of devices during use at a medical scene. The processing circuitry may be configured to obtain at least one series of master times, where each series of the at least one series of master times represents timestamps of a respective master device of at least one master device of the number of devices, and each master time of each series of the at least one series of master times corresponds to issuance of a timing message for one or more other devices of the number of devices. The processing circuitry may be configured to identify, based in part on each respective time series of the at least one series of master times, one or more time differences between the master device of the respective time series and each device of the one or more other devices, where identifying the at least one time difference includes i) for each respective device of at least a portion of the one or more other devices, identifying, in the data set of the respective device, at least one fiducial marker, each fiducial marker representing a respective timing difference of one or more timing differences between the respective device and the master device at a respective registration time of the fiducial marker, and/or ii) for each respective device of at least a portion of the one or more other devices, identifying, in a timing log of the respective device including one or more time entries, a respective timing difference of at least one timing difference between the respective device and the master device at a respective time of entry of each respective time entry of the timing log. The processing circuitry may be configured to apply the at least one time difference to align each data set of the number of data sets corresponding to the one or more other devices with the series of master times.

In some embodiments, the non-volatile data archive is distributed across a number of cloud-based storage devices. The system may include a network communications interface, where each data set of the number of data sets was uploaded to the system via the network communications interface.

In some embodiments, the at least one series of master times includes a first series of master times corresponding to a first master device and a second series of master times corresponding to a second master device, and the processing circuitry is further configured to identify at least one time difference between the first master device and the second master device. Identifying the at least one time difference between the first master device and the second master device may include identifying a first time difference between a given device of the number of devices and the first master device, identifying a second time difference between the given device and the second master device, and calculating an offset between the first time difference and the second time difference. The processing circuitry may be further configured to apply the at least one time difference to the second series of master times. The second time series of master times may represent a time period after a prior time period of the first time series of master times, and applying the at least one time difference to align each data set of the number of data sets with the series of master times may include applying the at least one time difference between the first master device and the second master device to adjust the second series of master times.

In some embodiments, the number of data sets relate to one patient. The number of data sets may relate to two or more patients. The two or more patients may include a pregnant patient and a fetus.

The foregoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A through FIG. 1C illustrate schematic diagrams of example emergency care scenes in accordance with some embodiments;

FIG. 2A is a flow chart of an example method for capturing time logs for aligning data timing of data sets collected by devices used at a medical scene;

FIG. 2B is a flow chart of an example method for sharing master time information with devices used at a medical scene for aligning data timing of data sets collected by the devices;

FIG. 3 is a block diagram of an example platform and architecture for aligning data timing of data sets collected by devices used at a medical scene;

FIG. 4A is an example time log that may be captured by a master timing device at a medical scene;

FIG. 4B is an example time log that may be captured by a device at a medical scene that is receiving timing synchronization information from a master timing device;

FIG. 5A and FIG. 5B present a graphical example of misaligned data that is brought into alignment in accordance with some embodiments;

FIG. 5C and FIG. 5D illustrate example misaligned data including a timing drift and application of skew adjustment during alignment;

FIG. 5E presents a graphical example of devices receiving timing synchronization information from a master timing device in accordance with some embodiments;

FIG. 6 is a flow chart of an example method for aligning data timing of collected data sets;

FIG. 7A and FIG. 7B illustrate medical devices collecting data sets during emergency care of a patient while in transit to a medical care facility and at the medical care facility; and

FIG. 8 is a block diagram of an example system of computing devices configured for use at an emergency medical scene.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

Aspects of the present disclosure are directed to systems and methods for aligning and synchronizing data for post-case review and/or real-time during a medical event collected by one or more medical treatment devices during the emergency medical treatment, such as by coordinating timestamps applied to such data. In some embodiments, the current time as understood by each device is logged in correspondence with the current time as understood by a master timing device such that timestamped data entries generated by each device may be aligned with a same clock through determining an offset between the master timing device and each device. The corresponding times may be logged periodically as timing markers, for example to allow for adjustment due to drift in the timing offsets. Aspects of the present disclosure provide practical solutions to correct what would otherwise result in misaligned data in a patient case file, where different types of data are originated from multiple sources at a medical rescue scene. Such misalignment may be due to a number of issues, for example, offset internal device clocks, skew in the internal device clocks relative to one another, and/or other causes of error.

In some implementations, the master timing device collects the current time as understood by each device and uses the current times to create timing markers. The current times may be issued in wired or wireless communications between the master timing device and each device, depending on a connection between each device and the master timing device. In other implementations, each device collects the current time as understood by the master device and creates a locally stored timing marker using the master device's current time. The current times may be issued in wired or wireless communications between the master timing device and each device, depending on a connection between each device and the master timing device.

In some embodiments, the medical treatment devices are collecting data in an emergency medical services (EMS) environment, where devices are used by trained emergency responders. In one example, the data may be collected during a rescue effort at the scene of an emergency or during prehospital transport. In another example, the data may be collected during ground or air transport of a patient between medical facilities. In a further example, the medical treatment devices may be collecting data regarding the status of a patient and/or one or more therapies delivered to a patient in a hospital emergency room, general medical-surgical and intermediate care floors, cardiac care unit, electrophysiology (EP) lab, operating rooms, and other similar areas of the hospital and/or for intra-hospital transport of patients.

In an example scenario, during the course of treating a cardiac arrest victim (a “code”), an emergency department (ED) medical caregiver personnel such as a nurse or physician brings in a medical treatment device such as a defibrillator/monitor and applies electrocardiographic (ECG) diagnostic electrodes and/or therapeutic defibrillation electrodes to the patient along. Further, the medical caregiver personnel may connect other physiologic sensors to the patient such as, in some examples, pulse oximetry, capnography, blood pressure, and/or near infrared spectroscopy monitors to collect physiological data regarding the patient. In addition, in certain situations, sensor data may be collected by one or more devices measuring caregiver performance, such as motion and flow sensors that measure caregiver performance of both diagnostic and treatment activities. For example, a motion sensor (e.g., accelerometer) may be placed on the patient's sternum during chest compressions and may be used to measure parameters indicative of chest compression performance, such as compression depth, rate, release velocity, amongst others; and/or a flow sensor may be placed along the patient's airway during ventilation and may be used to measure parameters indicative of ventilation performance, such as tidal volume, ventilation rate, etc.

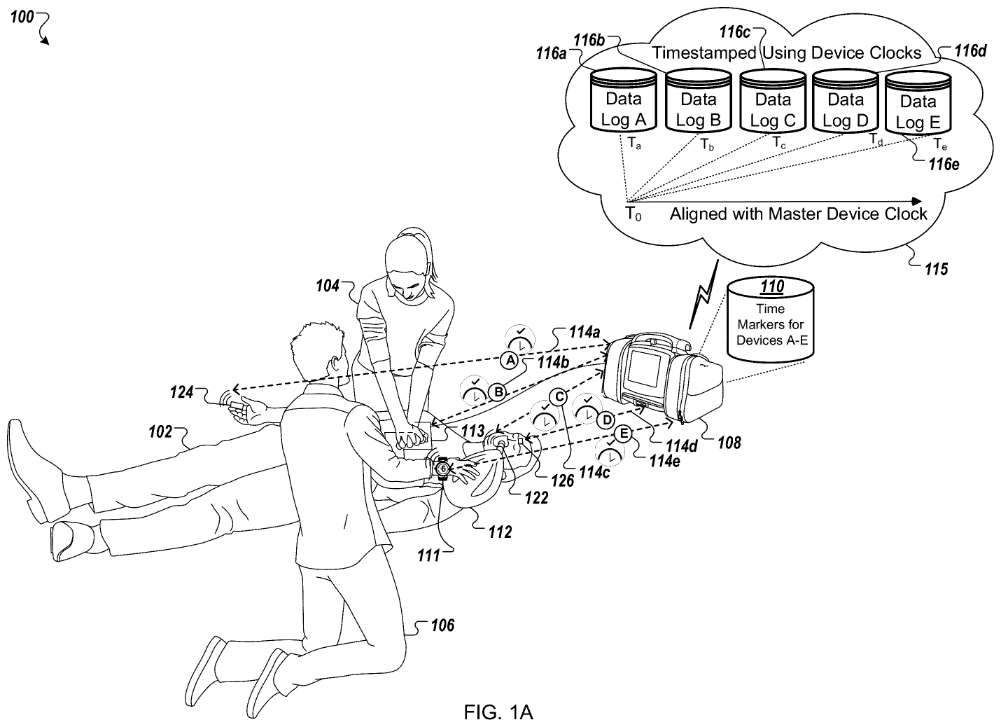

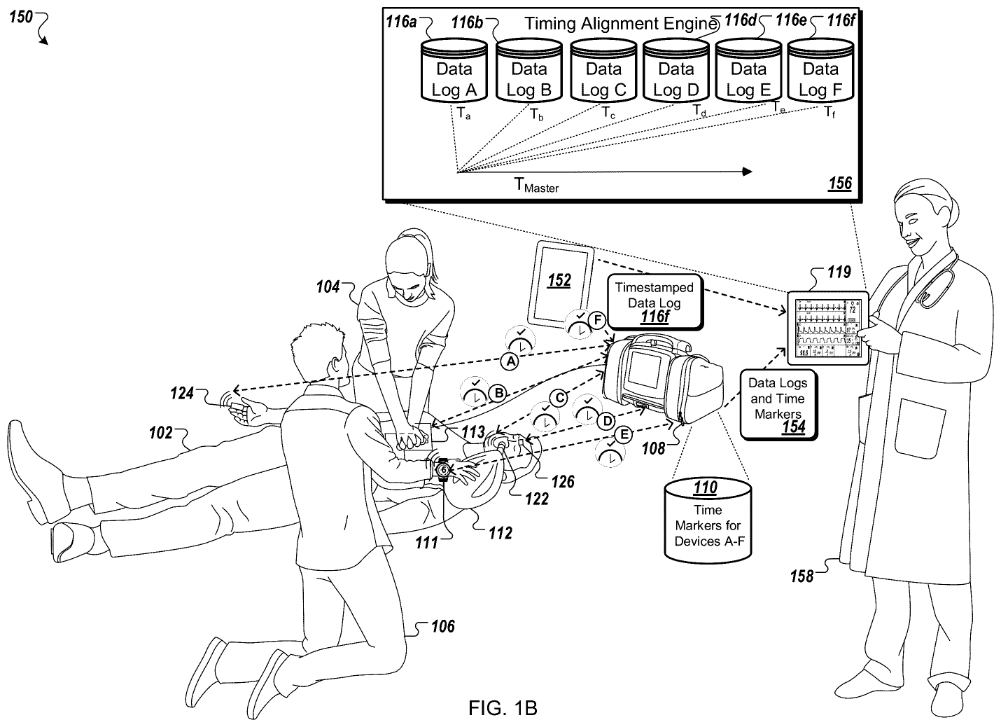

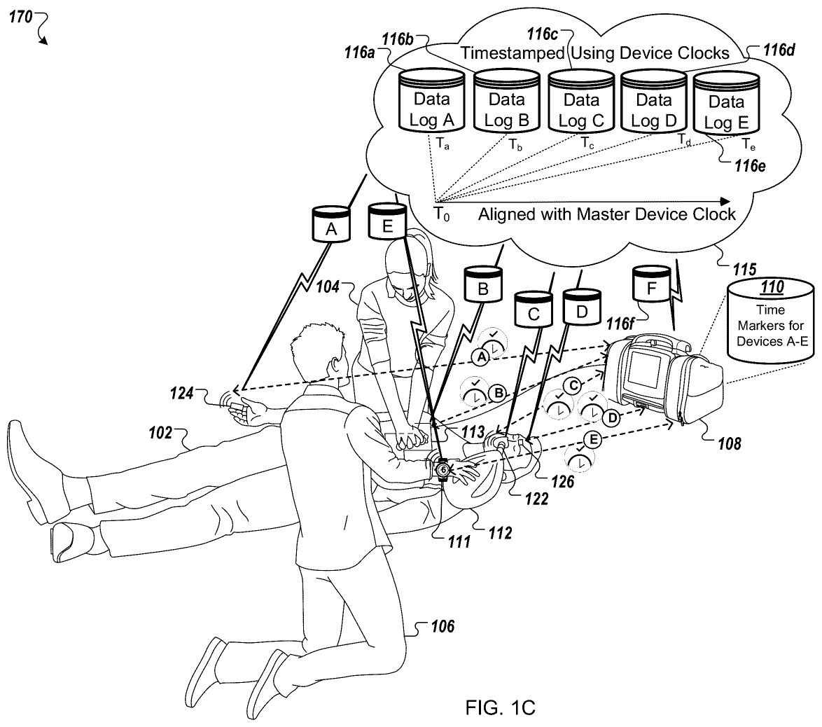

Referring to FIG. 1A, at an emergency care scene 100, a rescuer 104 performs cardiopulmonary resuscitation (CPR) on a victim or patient 102 (the terms are used interchangeably here to indicate a person who is the subject of intended or actual CPR and related treatment, or other medical treatment), such as an individual who has apparently undergone sudden cardiac arrest. The emergency care scene 100 can be, for instance, at the scene of an accident or health emergency, in an ambulance, in an emergency room or hospital, or another type of emergency situation. The rescuer 104 can be, for instance, a civilian responder with limited or no training in lifesaving techniques; a first responder, such as an emergency medical technician (EMT), police officer, or firefighter; or a medical professional, such as a physician or nurse. The rescuer 104 may be acting alone or may be acting with assistance from one or more other rescuers, such as a partner EMT 106. In the example of FIG. 1A, the rescuer 104 can deliver chest compressions to the patient 102 and the rescuer 106 can deliver ventilations to the patient 102 using a ventilation device 112 (e.g., bag-valve mask). The ventilation device 112 may be a portable ventilator that can be used in the hospital and/or pre-hospital environments such as aeromedical and ground transport, mass casualty situations, and extreme environments. In one example, the ventilation device 112 may weigh less than 10 pounds to provide for easy transport at emergency scenes so that pre-hospital ventilation can be delivered to a patient.

In this illustration, the rescuers 104, 106 can deploy a defibrillator 108, such as an automated external defibrillator (AED), a professional defibrillator, or another type of defibrillating apparatus, to treat the patient 102. The defibrillator 108 is connected to electrode pads 113 intended to be placed on the patient's chest via one or more cables. The electrode pads 113 may acquire signals indicative of the patient's ECG and the defibrillator 108 may analyze those signals and, if the signals determine that the patient is in need of defibrillation, provide defibrillation treatment to the patient 102 as appropriate through the electrode pads 113. In some examples, the defibrillator 108 can instruct one or more of the rescuers 104, 106 in providing CPR or other treatment to the patient 102.

The rescuers 104, 106, in some implementations, use companion devices such as smartphones, tablets, or wearable devices (e.g., watches or glasses) to assist in treating the patient 102. As illustrated, rescuer 106 is wearing a smart watch style companion device 111, which can provide prompting to assist a rescuer in delivering chest compressions, ventilations, mouth-to-mouth resuscitation, defibrillation, and/or other treatments to the patient 102.

One or more sensors (e.g., sensors 122, 126), in some implementations, are used to monitor the patient 102. For instance, the sensors 122, 126 monitor parameters indicative of the patient's health status. The sensors 122, 126 may monitor physical parameters such as the patient's heart rate, electrocardiogram (ECG), blood pressure, temperature, respiration rate, blood oxygen level, end-tidal carbon dioxide level (ETCO2), pulmonary function, blood glucose level, and/or other parameters indicative of the patient's health status. Some sensors, such as heart rate or ECG sensors, can be included in pads of the defibrillator 108. One or more sensors monitor the treatment delivered to the patient 102. For instance, a compression puck can be positioned beneath the hands of rescuer 104 as the rescuer 104 administers CPR by detecting a rate, depth, or duration of compressions delivered to the patient 102. Additionally, an airflow sensor 122 on a ventilation device 112 can monitor volume and rate of ventilations administered to the patient 102 by rescuer 106. Some sensors can monitor both parameters indicative of the patient's health status and parameters indicative of the treatment delivered to the patient. For example, ventilation sensors 122 can provide information about the patient's health status or information about the treatment delivered to the patient 102 to the defibrillator 108, one or more of the companion devices or other computing devices at the emergency care scene 100 or to remote computing devices such as one or more cloud servers of a cloud computing environment 115 that host data storage or web portals for the medical treatment system. A pulse oximeter sensor 124 can provide signals regarding blood oxygen levels in the patient 102. A temperature sensor 126 can provide signals regarding patient temperature.

In some implementations, each of the sensors or medical devices is configured to coordinate timing information via wired and/or wireless communication signals with a medical device or computing device acting as a master device. As illustrated, the defibrillation device 108 is a master timing device, receiving timing indications 114 from each of the patient sensor devices 122, 124, and 126, as well as devices 111 and 113. The timing indications 114, for example, may be provided via a wireless communication protocol such as, in some examples, Bluetooth, Wi-Fi, or Zigbee. As illustrated, the electrode pads 113 are in wired communication with the defibrillator 108.

In some embodiments, the timing indications 114 are provided to the master timing device responsive to a request issued by the master timing device. Issuing the request, in one example, can include communicating a broadcast message issued to any sensors and/or devices capable of receiving the message via the communications medium (e.g., wired or wireless). In another example, issuing the request can include individually issuing requests directed individually to one or more sensors and/or devices (e.g., over a private communication channel and/or in a manner in which only a particular recipient is capable of reading the request). Issuing a broadcast message may provide the benefit of reducing complexity of the signaling system, while issuing individual requests can provide a greater level of security in the messaging. Requests may be issued once every one to five minutes. Frequency of the requests, in some examples, may be based in part on responsiveness of the sensors and/or devices to the request messages (e.g., whether or not information is provided in response each round), a power level of the master timing device (e.g., more infrequent broadcasts may be provided in part to conserve energy), and/or power consumption concerns regarding one or more sensors and/or devices (e.g., to avoid depleting battery of various battery-operated sensors and/or devices). Further, the frequency may be determined in part to avoid processing lags in any of the devices, such that the primary function of each sensor or device goes uninterrupted or suffers minor interruption related to handling the timing requests.

The master timing device, in some embodiments, is configured to log information regarding the timing indications 114 in a time marker data set 110. The information, for example, may include a device identifier uniquely identifying the source device and/or sensor, a current device time according to the device or sensor, as issued in each timing indication 114, and/or a receipt time (e.g., corresponding master clock time) as understood by the master timing device.

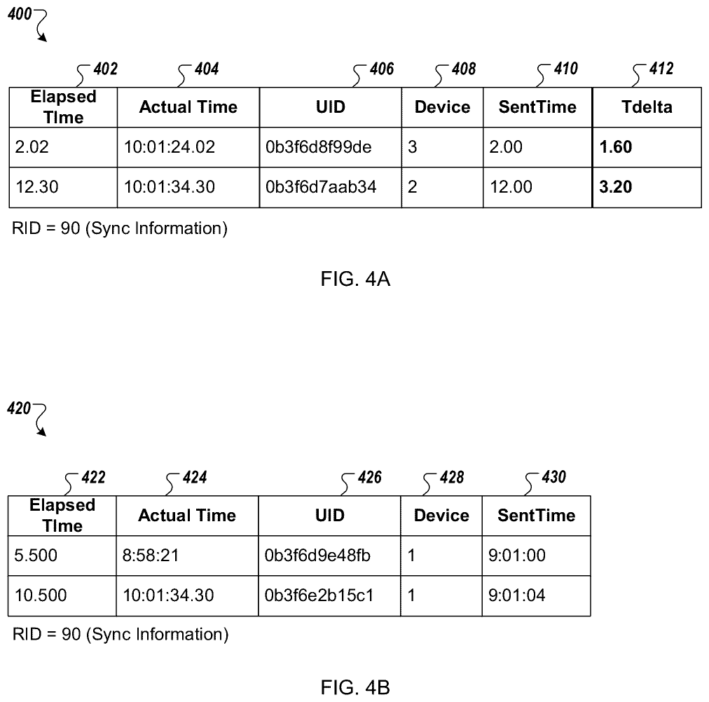

As shown in FIG. 4A, an example time log 400 (e.g., record identifier 90, pertaining to time synchronization information) includes an elapsed time 402 recording seconds that have elapsed since the beginning of treatment (e.g., the start of the device that is capturing the time log 400 behaving as the master device), as well as an actual time 404 recording the clock time of the master device and a sent time 410 recording the elapsed time 402 at which the synchronization request was issued by the master device. As illustrated, the elapsed time 402, the actual time 404, and the sent time 410 are each recorded to hundredths of a second, illustrated by way of example. The device time and the master clock time, in some embodiments, are logged at one second granularity, half second granularity, or tenth of a second granularity. That is, each of the respective times may be recorded to greater or less precision, for example, to the tenths of a second, to the hundredths of a second, to the thousandths of a second, to the ten thousandths of a second, and so on. The sent time 410, in some embodiments, is recorded to adjust for time of flight of communications between the master device and other devices (e.g., identified using device identifiers 408). The example time log 400 additionally includes, in some embodiments, a unique message identifier (UID) 406 corresponding to each response message.

In some implementations, as illustrated, the master device records a time delta 412 capturing a difference between the time as understood by the corresponding device 408 and the actual time 404 of the master device. The time delta 412, for example, may be directly applied for aligning data logs. In other embodiments, the clock time and/or elapsed time as understood by each device 408 are recorded in the time log 400.

Returning to FIG. 1A, the time marker data set 110, for example, can be stored as one or more data logs, such as a database file, comma separated value (CSV) file, and/or data table. In another example, a portion of the time markers 110 may be embedded in data collected from each corresponding device 111, 113, and/or sensors 122, 124, and/or 126 also transmitting data to the master timing device (e.g., the defibrillator 108). For example, one or more of the timing indications 114 may be provided along with a current sensor data value or other information collected by that device or sensor.

The collected data, in some embodiments, is transferred eventually to the cloud computing environment 115 as a set of data logs 116. The master timing device 108, for example, may collect data from each of the devices or sensors as the set of data logs 116a-e and, when connected to a network, may provide the data logs 116a-e for timing alignment and analysis. Certain devices and/or sensors, in another example, may directly or indirectly upload data to the cloud computing environment 115 without transmission to the defibrillator 108. The upload(s) may occur periodically during treatment of the patient 102, ongoing during treatment, and/or post-treatment. The data may be transferred to a separate device, such as an edge server or medical facility server, prior to being transferred to the cloud computing environment 115. In an illustrative example, upon connection to a computing device such as a laptop computer, tablet computer, or smart phone, the data log 116e collected by the smart watch style companion device 111, may be transferred to the computing devices which, in turn, may transfer the data log 116e to the cloud computing environment 115.

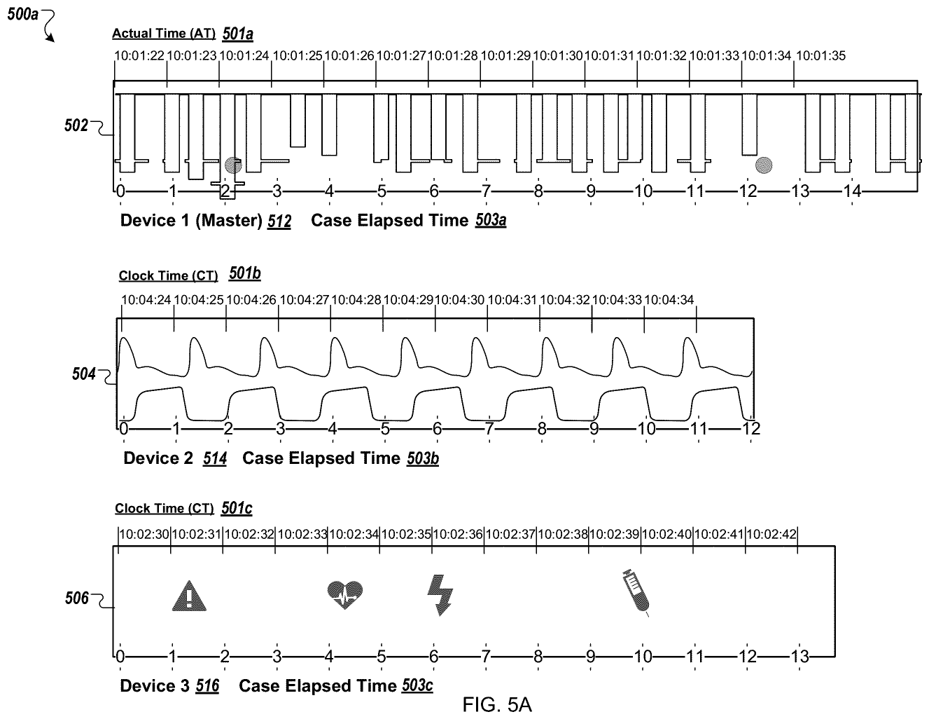

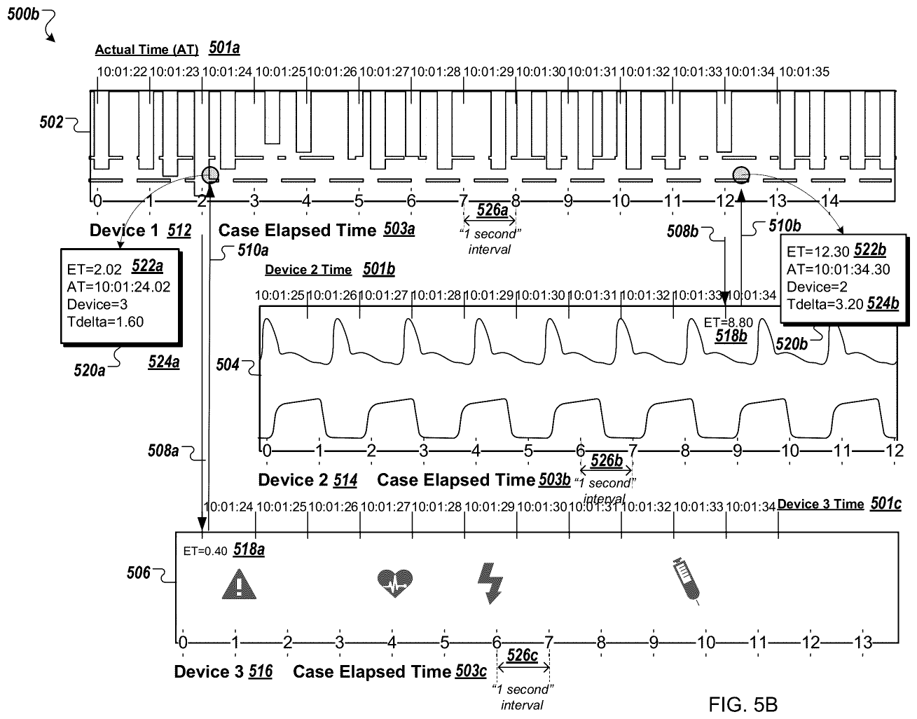

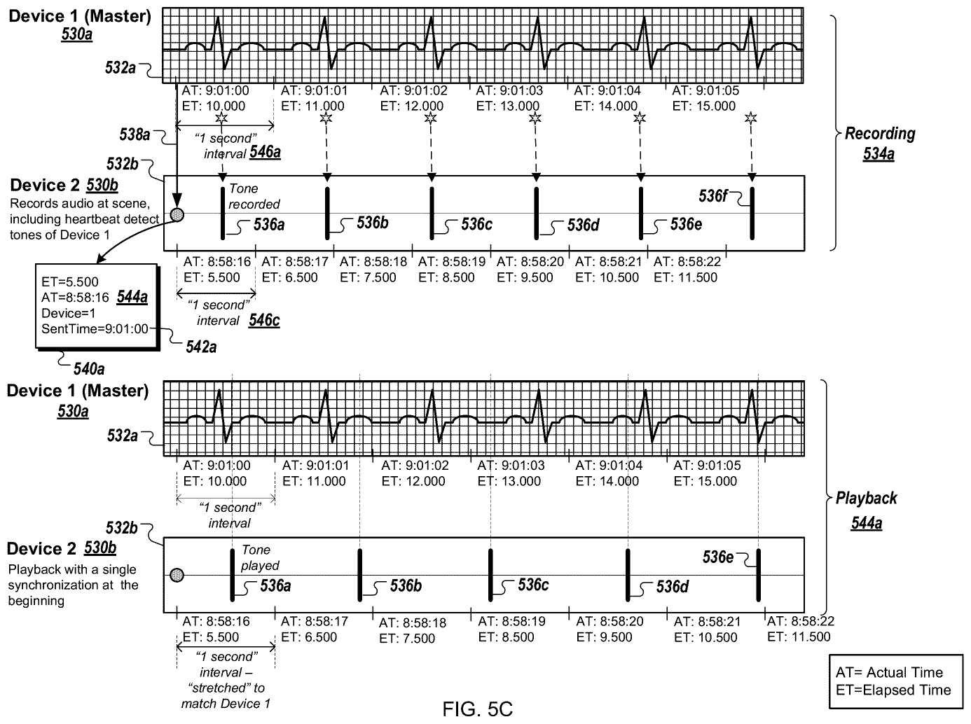

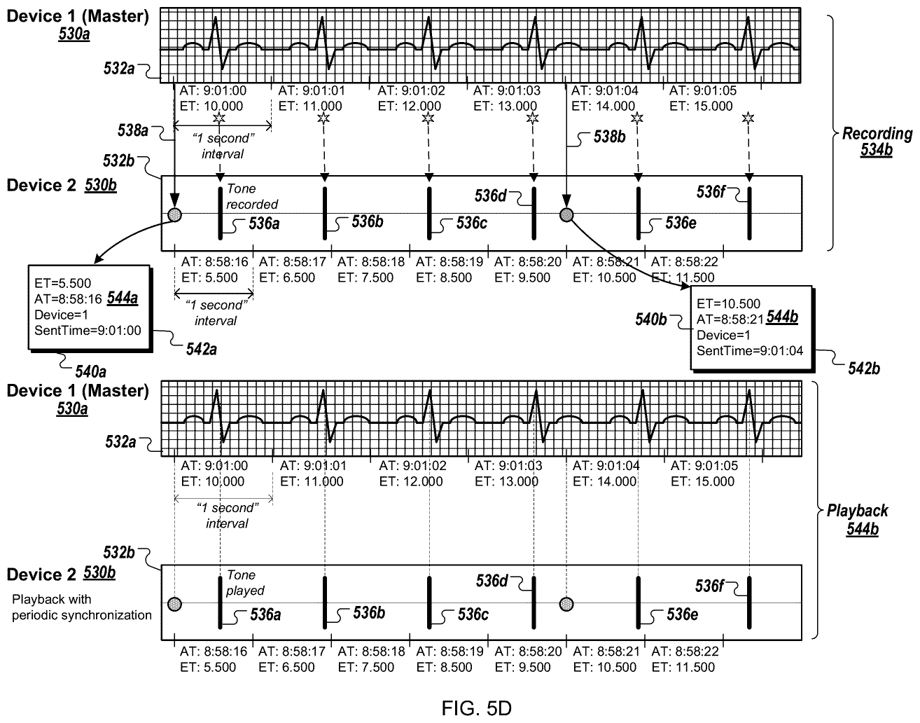

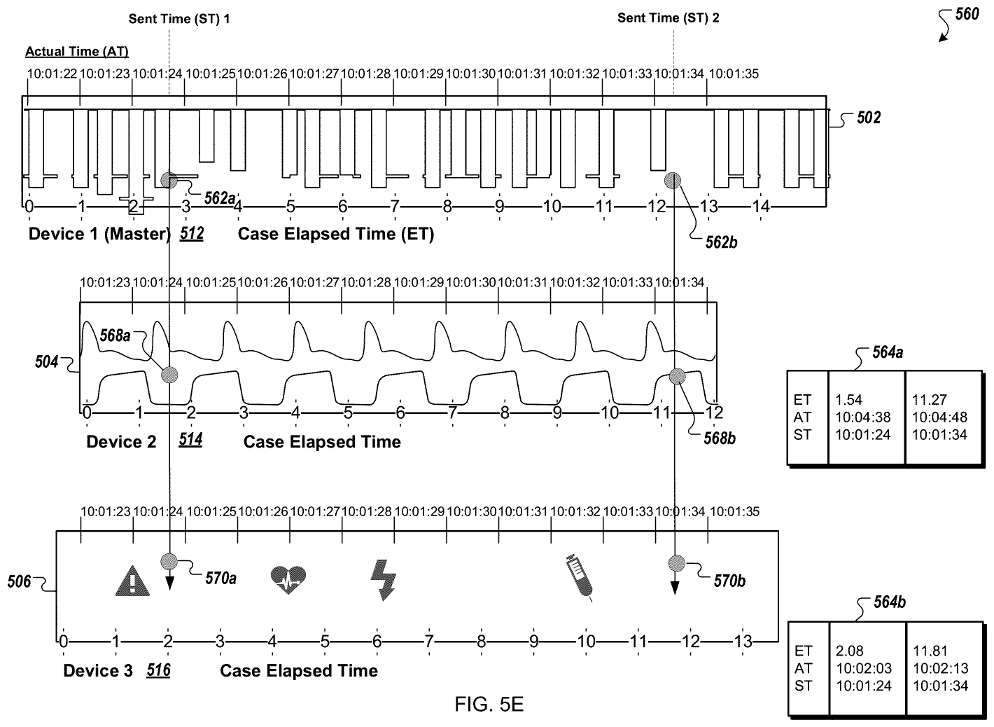

At the cloud computing environment 115, in some implementations, the time markers 110 are used to align the timing of timestamped data values captured in each of the data logs 116a-e. As illustrated, the correlations captured in the time markers 110 allow alignment of the data values of each data log 116a-116e with the timestamp of the master device clock using an offset between the device time transmitted in the timing responses 114 and the time of the clock of the defibrillator 108 at time of receipt. By using the offsets to align data retrieved from the data logs, graphical analyses of sensor data or other event data captured by the various devices and sensors may be presented in a single display for a holistic review of physiological events and treatment events that occurred during treatment of the patient 102. For example, as illustrated in FIG. 5A and FIG. 5B, a review display may include compression data 502, ECG data 504, and intervention event data 506. Further, in some embodiments, aggregate metrics involving physiological data captured from multiple sensors may be calculated and analyzed through aligning the timings of the various sensor data logs 116.

Turning to FIG. 1B, in some implementations, a scene 150 illustrates real-time or near real-time data alignment of information provided to a companion device 119 by the sensors and/or devices 111, 113, 122, 124, and 126 at the scene 150. As illustrated, a supervisor 158 can use the companion device 119 (e.g., tablet computer) to coordinate treatment provided by the multiple rescuers 104, 106. The supervisor 158, for example, may oversee and coordinate patient treatment during a critical patient care event (e.g., chest pain, cardiac arrest, traumatic brain injury (TBI), respiratory distress, or critical care monitoring). Further, the scene 150 may include another computing device 152 (e.g., smart phone, tablet computer, etc.) configured to provide a timestamped data log 154 to the companion device 119. The computing device 152, for example, may be used by one of the rescuers 104, 106 to verbally and/or manually log intervention information, such as the timing and dosage of medications. In another example, the computing device 152 may present feedback particular to the role of a given rescuer. For example, the companion device 152 may display chest compression related metrics, ventilation delivery related metrics, and/or processed ventilator sensor data such as airway pressure (PAW), plateau pressure (PPlat), peak inspiratory pressure (PIP), or fraction of inspired oxygen (FIO2). The companion device 119 may present overview information related to the activities at the scene 150 in general.

In some implementations, the companion device 119 presents waveforms, such as ECG waveforms, as well as numerical values representing non-continuous physiological measurements (e.g., NIBP, SpO2, HR, EtCO2). The companion device 119, in one mode, may display a visual reproduction of the information displayed at the defibrillator 108. For example, the supervisor 158 may view, in real-time, the same waveforms and patient data displayed at the defibrillator 108.

In some embodiments, the companion device 119 presents waveforms, data dashboards, and/or other information that are pertinent to a particular type of care being delivered to the patient 102. A “basic monitoring” view, for example, may display ECG and SPO-2 waveforms as well as heart rate, blood pressure, and SPO2 trends. In another example, a TBI view may display ECG, ETCO2, and SPO-2 waveforms, heart rate and respiratory rate trends, and a TBI dashboard. In a third example, an “advanced monitoring” may display ECG, ETCO2, and SPO-2 waveforms and heart rate, 12-lead results, ST trending, blood pressure, SPO2, ETCO2, and respiratory rate trends. In a fourth example, a “respiratory distress” view may display ECG, ETCO2, and SPO¬2 waveforms, heart rate, blood pressure, SPO2, ETCO2, and respiratory rate trends, and a ventilation (BVM) dashboard. In a fifth example, a “cardiac arrest” view may display ECG and SPO¬2 waveforms, SPO2 and ETCO2 trends, and CPR and ventilation dashboards. In a sixth example, a “critical care monitoring” view may display ECG, ETCO2, SPO¬2, and invasive blood pressure (IBP) 1/2/3 waveforms and heart rate (HR), respiratory rate (RR), NIBP, ETCO2, SPO-2, and IBP1/2/3 trends.

In some implementations, the companion device 119 presents data trends during a medical event, such as physiological sensor value trends (e.g., ST trending, SPO-2, ETCO2, blood pressure, heart rate) derived from sensor data captured over time during the medical event. The trend data may be displayed in a graphical or tabular format.

In some implementations, the companion device 152 is used by the rescuers 104, 106 at the scene 150 to enter information regarding treatment provided to the patient 102. The rescuers 104, 106, for example, may enter patient data (e.g., name, gender, age, birth date, etc.), medicine delivery data (e.g., medicine type, time provided, dose provided, etc.), and/or other therapy delivery data (e.g., IV bag, etc.). The information regarding treatment, for example, may be included in a timestamped data log 116f. The data log 116f may be maintained by the device 152 or provided to the companion device 119.

In some embodiments, a portion of the information entered into the companion device 152 is provided verbally. The companion device 152 (and/or a separate computing device such as an edge server), for example, may perform natural language processing (NLP) on verbal timing markers related to the provision of medicine or other therapy and register the verbally inputted time markers in the timestamped data log 116f.

In some implementations, the companion device 119 receives the timestamped data log 116f from the companion device 152 as well as data logs and time markers 154 from the defibrillator 108 (e.g., containing the time markers 110 as well as data logs 116a-e from the sensors 122, 124, 126 and devices 111, 113). The data logs 116a-f, for example, may be provided to the companion device 119 as individual real-time or near real-time data streams from the defibrillator 108 and the companion device 152. Further, since the devices 111, 113, sensors 122, 124, 126, defibrillator (e.g., master timing device) 108, and companion devices 119, 152, are all in close proximity, to perform real-time or near real-time data alignment, the device time and the master clock time may be logged at a 100 millisecond, 10 millisecond, or one millisecond granularity.

In some implementations, the speed of connections between the devices 111, 113, sensors 122, 124, 126, defibrillator (e.g., master timing device) 108, and companion devices 119, 152 may determine, in part, the granularity of timing used in aligning the data. The devices 111, 113, sensors 122, 124, 126, defibrillator (e.g., master timing device) 108, and companion devices 119, 152, for example, may be in direct wireless communication (e.g., without communication via a local area network (LAN), wide area network (WAN), the Internet, a cloud service, or other intervening network communication). In some examples, the direct wireless communication may be provided by one or more of Wi-Fi, Bluetooth, near field communication (NFC), or Zigbee. The wireless communication channel, in some implementations, is a secure channel. For example, the wireless communication channel may be password protected, encoded, encrypted, or otherwise protected from connection by unauthorized devices.

In some implementations, the companion device 119 executes a timing alignment engine 156 to align the data in the data logs 116a-116f. The aligned data may be used to present combined metrics, graphics, and/or other analysis on the companion device 119 for the attention of the supervisor 158.

Although the companion devices 119, 152 are presented as tablet computing devices, in other embodiments, computing devices such as laptop computers or computing devices integrated into an ambulance may be used to collect the data logs and time markers 154, enabling real-time analysis of health data about the patient 102 or data indicative of treatment delivered to the patient 102 as well as communication of such data to a remote location (e.g., a dispatch center, an emergency room, or a remote server) for review by other medical personnel.

FIG. 1C illustrates a medical emergency scene 170, similar to the scene 100 of FIG. 1A. As illustrated in the scene 170, each sensor 122, 124, 126 and each device 111, 113 uploads its corresponding data log 116a-116e to the cloud computing environment 115. Further, the defibrillator 108 uploads a data log 116f as well as the time markers 110 to the cloud computing environment 115 separately. In the cloud computing environment 115, one or more offsets to apply to the timestamps of each of the data logs 116a-116e is calculated based on the time markers 110, effectively aligning data from the data logs 116a-116e with the timing of the data log 116f of the defibrillator 108.

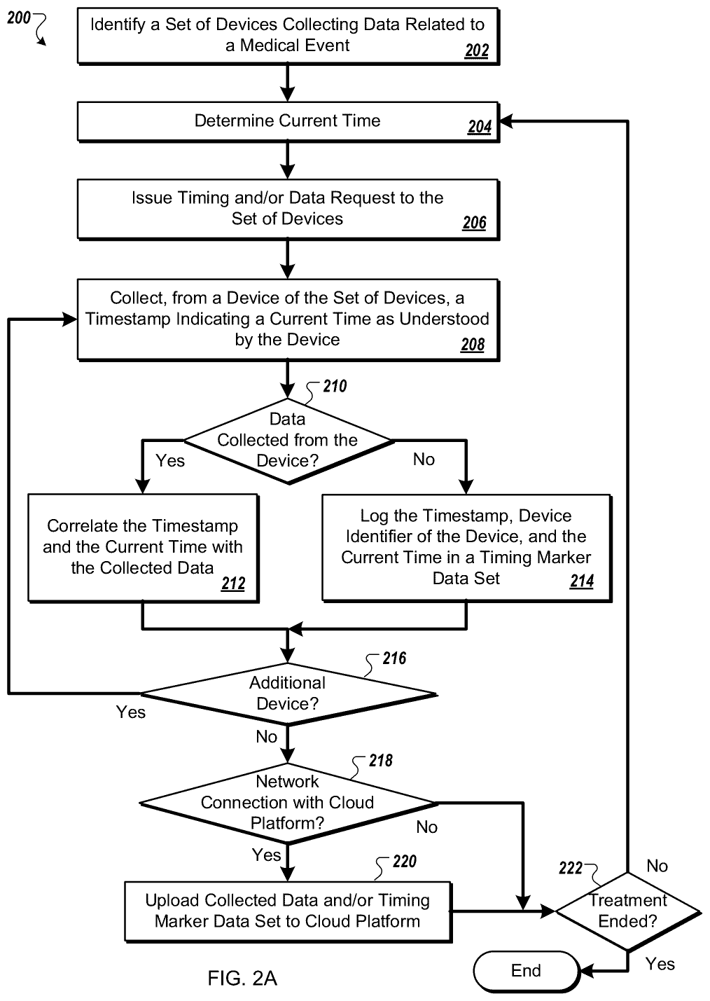

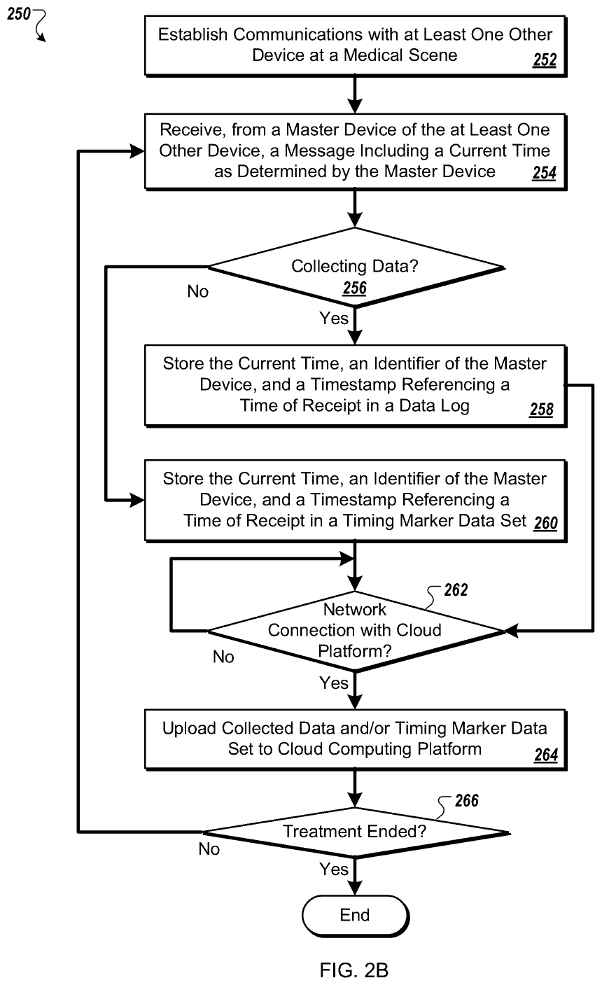

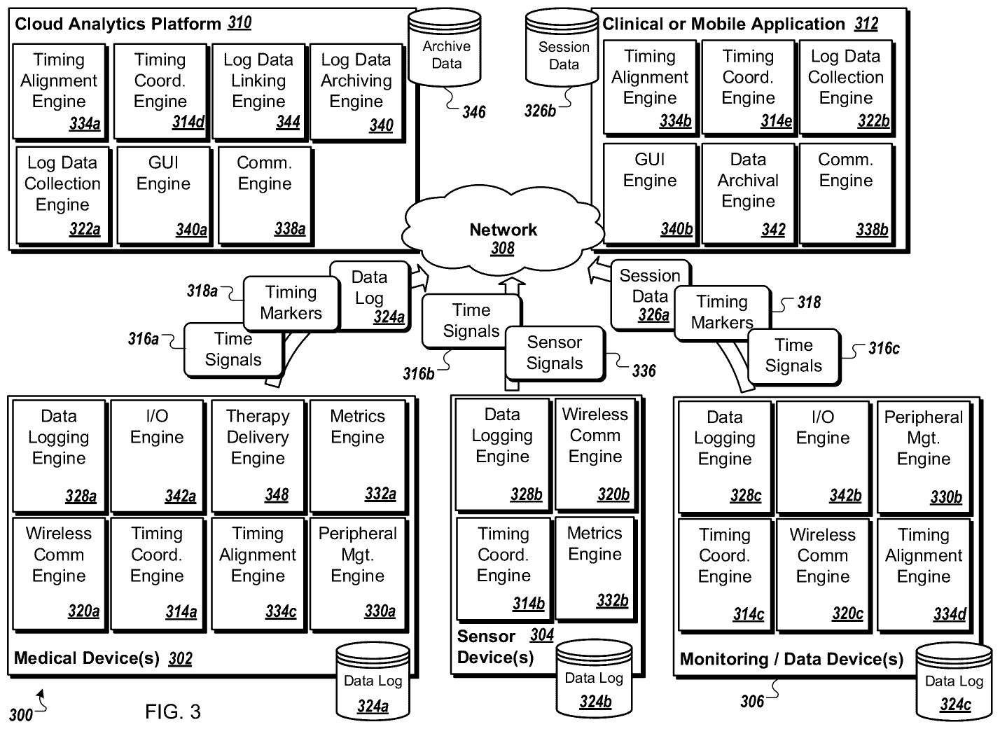

FIG. 2A is a flow chart of an example method 200 for capturing time logs for aligning data timing of data sets collected by devices used at a medical scene. The method 200 may be performed by a master timing device, as described in relation to FIG. 1A through FIG. 1C. Portions of the method 200, in some examples, may be performed by the defibrillator 108 of FIG. 1A, the companion device 119 of FIG. 1B, and/or the cloud computing environment 115 of FIG. 1C. The method, in some embodiments, is performed by a timing coordination engine 314, described below in relation to FIG. 3. As illustrated in FIG. 3, for example, portions of the method 200 may be performed by a medical device 302, a sensor device 304, a monitoring and/or data device 306, a cloud analytics platform 310, and/or a clinical or mobile application 312, as illustrated in FIG. 3.

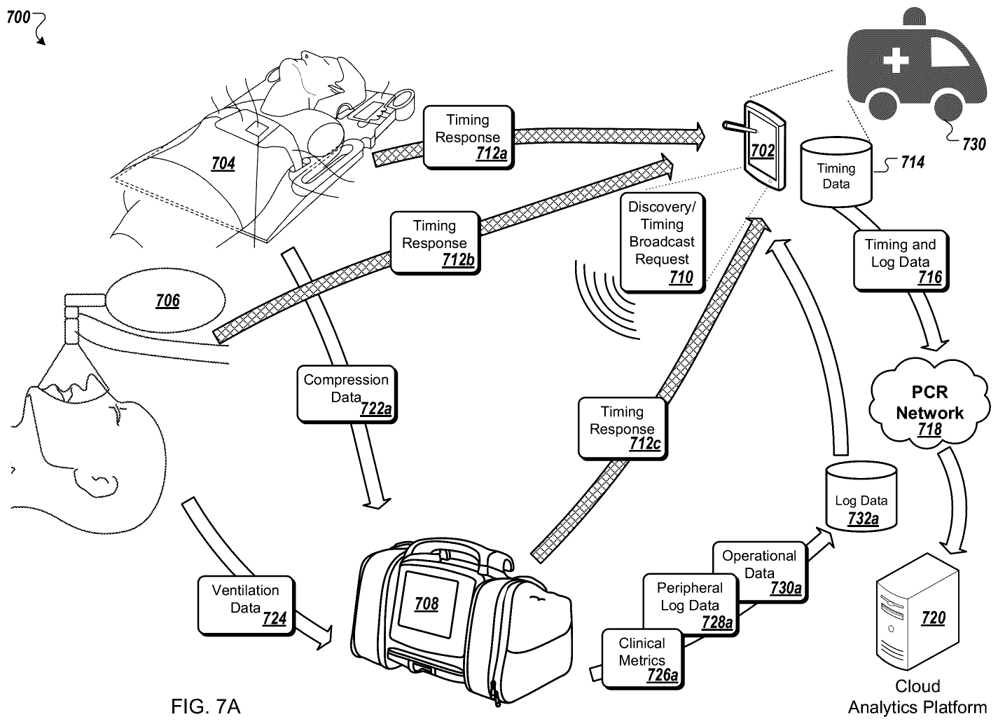

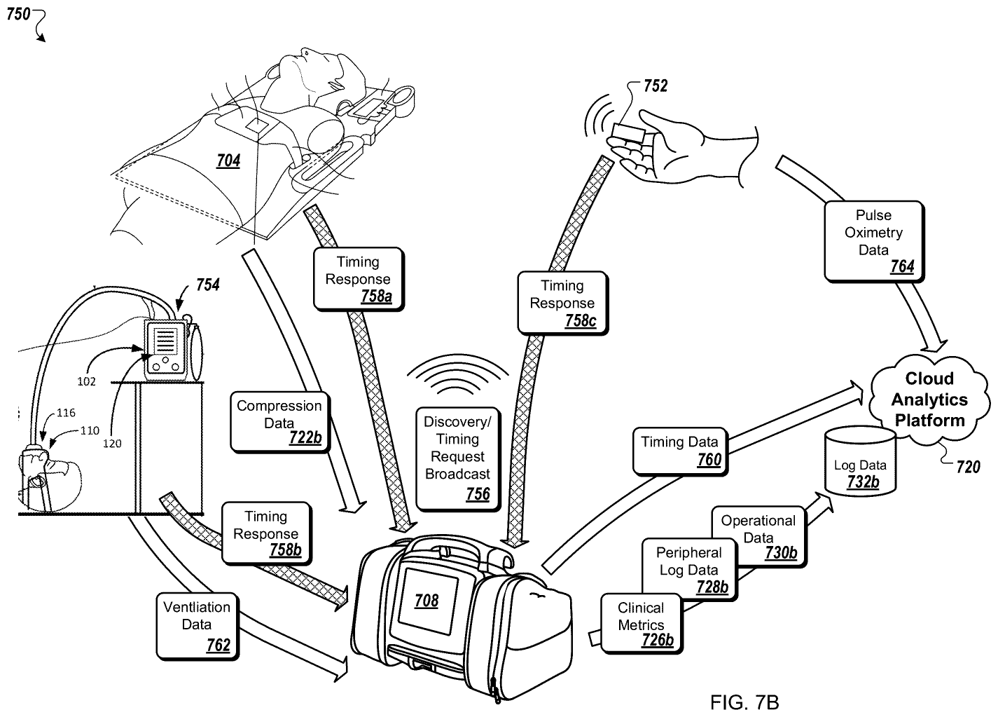

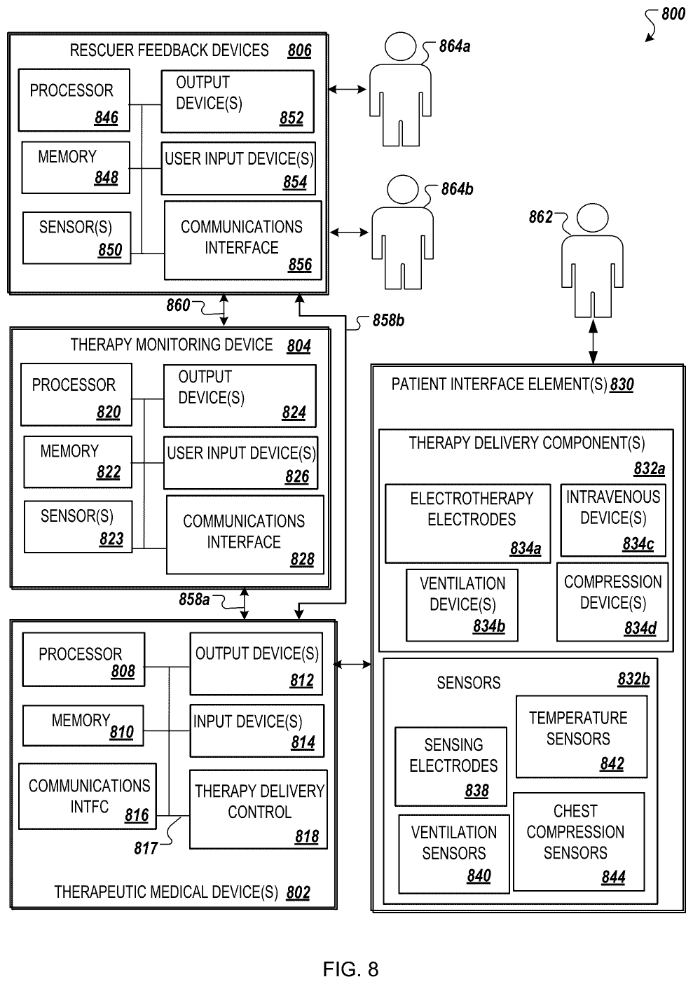

In some implementations, the method 200 begins with identifying a set of devices collecting data related to a medical event (202). One or more of the devices may be in wired communication such that a signaling handshake over the wired communication allows detection of the device. The set of medical devices may be proximate to (e.g., on scene at a medical event with) a device identifying the set of devices. For at least a portion of the set of devices, identifying may include detecting the presence of each device via a wireless communication network due to the device(s) being within wireless communication range. The wireless network, in some examples, may be a Wi-Fi, Bluetooth, or Zigbee network. Turning to FIG. 7A, a master device 702 (e.g., tablet computing device) may issue a discovery broadcast request 710 for receipt by nearby devices such as a compression device 704, a ventilation device 706, and a defibrillation device 708. In some embodiments, the master device 702 may serve as a companion device to a medical device such as a defibrillator/monitor, monitor, ventilator or other appropriate device. In other embodiments, the master device 702 may be independently functioning.

In some embodiments, a preconfigured pairing with one or more of the devices may have already been established, for example due to the devices having intercommunicated previously. The device may automatically connect, for example, due to the prior pairing (e.g., upon recognizing a wireless signal). Upon detection of each device, if not already preconfigured, the device may be authenticated prior to pairing. Authentication may involve a user entering information (e.g., credentials), such as, in some examples, username/password inputs, at least one biometric input provided at a biometric input interface (e.g., fingerprint, iris recognition, facial recognition), and/or a badge scan via a scanning sensor (e.g., RFID scan or a computer-readable code such as a QR code). Authentication may involve the device presenting a stored code or key for authentication.

In some implementations, a current time is determined (204). The current time, for example, may be determined by a master clock of the device executing the method 200. The current time may be determined from device synchronization with a Coordinated Universal Time (UTC) signal.

In some implementations, a timing and/or data request is issued to the set of devices (206). The request may include a timestamp having the current time. The request may be a broadcast message issued via wireless communications. For example, as illustrated in FIG. 7A, the timing broadcast request 710 may be issued for receipt by the compression device 704, the ventilation device 706, and the defibrillation device 708. The request, for one or more devices, may involve a point-to-point wireless communication between the master timing device and each device. The request may be a request for the current time as understood by each device. At least a portion of the set of devices, in some embodiments, include programming (e.g., as part of the timing coordination engine 314 of FIG. 3) for recognizing and responding to the timing request. In some embodiments, at least a portion of the devices are not programmed to coordinate timing with a master timing device. For example, to retrieve a current timestamp from a device not programmed to respond to specialized timing commands, a common handshaking mechanism that involves a timestamped response (e.g., such as an ICMP timestamp request) may be issued. In some embodiments, the request includes a request for data, such as a portion of a data log (e.g., data logs 116a-e of FIG. 1A). The request, for one or more devices, may involve issuing a request via wired communication with each device.

In some implementations, for each respective device of the set of devices, a timestamp indicating a current time as understood by the respective device is collected (208). The timestamps, for example, may be included in a response message to the request(s). As illustrated in FIG. 7A, for example, each of the compression device 704, the ventilation device 706, and the defibrillation device 708 issue a timing response 712 responsive to the timing broadcast request 710. The time provided, in some embodiments, has a granularity of at least one second. The granularity, in some examples, may include a half second, a tenth of a second, or a hundredth of a second. The granularity may differ from device to device, for example based on the programming of the device. Further, the granularity may depend in part on the communication protocol used for issuing the request to a particular device (e.g., wired or wireless, type of network, programmed to communicate via the timing coordination engine 314 or not, etc.). As illustrated in FIG. 3, the timestamps may be included in time signals 316 provided by one or more medical devices 302, sensor devices 304, and/or monitoring/data devices 306. The signals 316 may be issued via a wireless communication engine 320 of each of the medical devices 302, sensor devices 304, and/or monitoring/data devices 306.

In some implementations, if data has been collected from a given device (210), the timestamp and the current time are correlated with the collected data. For example, a time marker record may be embedded as part of the collected data log for the given device. The time marker record may include the timestamp and the current time, or the timestamp and an offset representing a difference between the timestamp and the current time.

Turning to FIG. 3, in some embodiments, a log collection data engine 322b of the clinical or mobile application 312 may collect data logs 324 and/or session data 326 from one or more medical devices 302 and/or monitoring/data devices 306. The data logs 324 may be produced by a data logging engine 328 of the medical device(s) 302 and/or the monitoring/data device(s) 306. The medical device(s) 302, for example, may include a defibrillator, a defibrillator/monitor, a mechanical ventilator such as the Z Vent® ventilator provided by ZOLL Medical Corporation, and/or another medical device configured to provide therapy to the patient via a therapy delivery engine 348. As another example, the medical therapy may be chest compression therapy for treatment of cardiac arrest and the medical device 302 may be a mechanical chest compression device such as a belt-based chest compression device or a piston-based chest compression device. In other examples, the medical therapy may be ventilation therapy, therapeutic cooling or other temperature management, invasive hemodynamic support therapy (e.g., Extracorporeal Membrane Oxygenation (ECMO)), etc. and the medical device 302 may be a device configured to provide a respective therapy. Further, in some embodiments, the medical device 302 includes a combination of one or more of these examples. The monitoring/data device(s) 306, in some examples, may include a portable computing device such as, in some examples, a laptop computer, tablet computer, mobile phone, or other handheld computing device. The therapy monitoring device(s) 306, in a particular example, may include the supervisor device 119 of FIG. 1B.

The monitoring/data device(s) 306 may collect data logs 324b from one or more sensor devices 304 in wired or wireless communication with the monitoring/data device(s) 306 using a peripheral management engine 330. The sensor devices 304, in some examples, may include electrotherapy electrodes including defibrillation electrodes and/or pacing electrodes, chest compression devices (e.g., one or more belts or a piston), ventilation devices (e.g., a mask and/or tubes), drug delivery devices, cardiac sensing electrodes, a chest compression sensor, ventilation sensors, conductive and/or capacitive electrodes configured to measure changes in a patient's electrophysiology such as ECG information, sensors for measuring physiological parameters such as vital signs, blood pressure (e.g., continuous or non-continuous), blood flow (e.g., via ultrasound Doppler), heart rate, pulse oxygen level, respiration rate, heart sounds, lung sounds, respiration sounds, end tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography. The session data 326, thus, may include data logs 324b collected by sensor devices 304 as well as the data log 324c of the monitoring/data device(s) 306. The sensor data log 324b may include raw signal data and/or data metrics. For example, some sensor devices 304 may include a metrics engine 332 for calculating metrics from sensor data.

In some implementations, if data is not collected from the given device (210), the timestamp, a device identifier of the device, and the current time may be logged in a timing marker data set (214). The timing coordination engine 314 of the medical device 302, monitoring/data device 306, or clinical/mobile application 312, for example, may log the information as timing markers 318.

The collecting (208) and correlating (212) or logging (214), in some implementations, repeat for each device (216).

In some implementations, if a network connection is available between the master timing device and a cloud computing platform (218), the collected data and/or timing marker data set is uploaded to the cloud computing platform (220). For example, the defibrillator 108 may upload the time markers 110 to the cloud computing environment 115 via the network 308 as illustrated in FIG. 1A. As illustrated in FIG. 3, for example, timing markers 318 may be provided by a master timing device that is either one of the medical devices 302 or one of the monitoring/data devices 306 to the cloud analytics platform 310 via the network 308. Further, the monitoring/data device(s) 306 may include time markers within the session data 326. As illustrated in FIG. 7A, the master timing device 702 may provide separate timing data 714 and/or timing and log data 716 to a cloud analytics platform 720 via a position-and-connectivity-based (PCR) network 718. The network access, for example, may be enabled by an ambulance 730 carrying or in communication with the master timing device 702, the compression device 704, the ventilation device 706, and the defibrillation device 708.

In some implementations, the method 200 repeats while treatment is ongoing (222).

Although illustrated as a particular series of operations, in some embodiments, the method 200 includes more or fewer operations. For example, upon collecting each timestamp, a latency offset may be applied based on communication latency between the device and the master timekeeper. Determining the latency offset may include performing a roundtrip or ping test involving rounds of simple communication with the device to establish an average or typical latency. In some embodiments, certain operations of the method 200 may be performed in a different order and/or concurrently. For example, timestamps may be correlated (212) and/or logged (214) while collecting (208) timestamps from other devices. Other modifications to the method 200 are possible while remaining in the spirit and scope of the method 200.

FIG. 2B is a flow chart of an example method 250 for sharing master time information with devices used at a medical scene for use in aligning data timing of data sets collected by the devices. Unlike the method 200 of FIG. 2A, in the method 250, individual devices in communication with a master timing device perform the method 250 to individually store timing fiducial markers for later aligning data with a master clock. The method 250, for example, will exhibit less latency since there is only a single communication path rather than a round-trip communication path. Further, the master device may save power and processing time, no longer needing to monitor for responses from each connected device and, potentially, re-send requests if one or more devices fail to respond. However, the method 250, if performed without round-trip communication, may have a downside in the master device not being configured to confirm that the request was received. Portions of the method 250, in some examples, may be performed by one or more of the sensor devices 122, 124, 126, the medical or computing devices 111, 113, and/or defibrillator 108 of FIG. 1A, and/or the companion device 119 of FIG. 1B. In some embodiments, the method 250 is performed by one or more of the medical devices 302, the sensor devices 304, and/or the monitoring/data devices 306 of FIG. 3. The method 250 may be used instead of, or in addition to, the method 200 of FIG. 2A.

In some implementations, the method 250 begins with establishing communications with at least one other device at a medical scene including a master timing device (252). Communications may be established, for example, as described in relation to operation 202 of the method 200 of FIG. 2A.

In some implementations, a message including a current time as determined by the master device is received from the master device (254). The message, for example, may be a timing and/or data request issued by the master device, for example as described in relation to operation 206 of FIG. 2A. The message may be a broadcast message not requiring a response. The message may include the timestamp and a device identifier corresponding to the master device.

If data is being collected (256), in some implementations, the current time, an identifier of the master device, and a timestamp referencing a time of receipt is stored in a data log (258). Certain devices, such as the medical devices 302, the sensor devices 304, and certain monitoring/data devices 306 of FIG. 3 may log data related to treatment of a patient at a medical scene (e.g., to data logs 324). When data is being logged for later analysis, the current time from the master timing device may be logged along with the data records. The data logging engine 328 of FIG. 3, for example, may log the timing fiducial marker to the data log 324.

In some implementations, if data is not being collected (256), the current time, the identifier of the master device, and the timestamp referencing the time of receipt is stored in a timing marker data set (260). Certain devices may collect data from other devices without actively capturing data related to the patient's treatment. For example, as described in relation to the monitoring/data devices 306 of FIG. 3, the device may collect data logs 324a,b from one or more medical devices 302 and/or sensor devices 304 as session data 326. In this circumstance, a separate timing markers data collection 318 may be captured by the monitoring/data devices 306. The timing coordination engine 314 of FIG. 3, for example, may log the timing fiducial marker to the timing markers 318.

In some implementations, if a network connection with a cloud computing platform is available (262), the collected data and/or timing marker data set is uploaded to the cloud computing platform (264). The network connection and upload, for example, may be performed as described in relation to operations 218 and 220 of FIG. 2A.

In some implementations, while patient treatment is ongoing (266), the method 250 repeats, for example as described in relation to operation 222 of FIG. 2A.

Although illustrated as a particular series of operations, in some embodiments, the method 250 includes more or fewer operations. For example, in some implementations, a response is issued to the master device upon receipt of the message. The response, for example, may include the timestamp referencing the time of receipt. In this manner, the device may be storing timing fiducial markers as a back-up in case a breakdown in communications causes the master device to fail to receive one or more responses from the device. In some embodiments, certain operations of the method 250 may be performed in a different order and/or concurrently. For example, fiducial markers may be collected in both the data log (258) and a timing marker data set (260) or, in other circumstances, timing markers are only stored to a timing marker data set (260) whether or not the device is actively logging data (256). Other modifications to the method 250 are possible while remaining in the spirit and scope of the method 250.

Turning to FIG. 5A and FIG. 5B, a series of block diagrams illustrate a process 500 for aligning an example data collection related to a medical scene, including compression data 502 of a first device 512, ECG data 504 of a second device 514, and therapy event data 506 of a third device 516. It can be appreciated that the data shown here are exemplary in nature and that aspects of the present disclosure can be applied to other embodiments where various types of data may be sourced from different devices. As illustrated in this particular example, the compression data 502 was collected by the first device 512 while acting as a master device for collecting timing logs from other devices at the medical scene.

Turning to FIG. 5A, three data sets, including a compression data set 502, an ECG data set 504, and a therapy data set 506, are illustrated in a first arrangement 500a. The data sets 502, 504, 506, as indicated, were captured by device 1 512, device 2 514, and device 3 516, respectively. The data sets 502, 504, 506, as illustrated, are aligned by an elapsed time 503a, 503b, 503c. However, without knowing when the case time initiated for each device (e.g., when each device was activated at the scene), the elapsed time 503 is not viable for use in aligning the data sets.

Further, as illustrated in FIG. 5A, a clock time 501a of the master device 512 (e.g., the “actual time” of the master clock which will be used for aligning purposes) differs from both a clock time 501b of the second device 514 and a clock time 501c of the third device 516. Thus, if each of the data sets 502, 504, and 506 were aligned by individual clock times, no alignment of the represented data portions would be possible.

Turning to FIG. 5B, in a second arrangement 500b, the data sets 502, 504, and 506 are aligned in accordance with synchronization records 520 captured by the master device 512. In some implementations, the first device 512 issues a synchronization request 508a to the third device 516 and receives a response message 510a. As can be seen, the third device 516 responds at a case elapsed time 518a of 0.40 seconds. As illustrated in a first synchronization record 520a, the first device 512 logs a timing delta 524a of 1.60 seconds, capturing a difference between an elapsed time (e.g., 2.0 seconds) as understood by the first device 512 when issuing the request message 508a and the elapsed time 518a as understood by the third device 516 when issuing the response 510a. The first synchronization record 520a may also include an elapsed time 522a corresponding to a time of receipt of the response message 510a. The information of the record 520a, for example, may correspond to information in the first row of the synchronization record 400 of FIG. 4A.

In some implementations, at a later time, the first device 512 issues a synchronization request 508b to the second device 514 and receives a response message 510b. As can be seen, the second device 514 responds at a case elapsed time 518b of 8.80 seconds. As illustrated in a second synchronization record 520b, the first device 512 logs a timing delta 524b of 3.20 seconds, capturing a difference between an elapsed time (e.g., 12.0 seconds) as understood by the first device 512 when issuing the request message 508b and the elapsed time 518b of 8.80 seconds as understood by the second device 514 when issuing the response 510b. The second synchronization record 520b may also include an elapsed time 522b corresponding to a time of receipt of the response message 510b. The information of the record 520b, for example, may correspond to information in the second row of the synchronization record 400 of FIG. 4A.

When aligning the ECG data 504 and the therapy data 506 with the compression data 502, the timing deltas 524a, 524b may be applied to synchronize the elapsed times according to each individual data set 502, 504, 506.

In some implementations, in addition to aligning due to clock offset, a skew may be introduced into the alignment due to the device clocks not running at the same rate of time (e.g., when separate internal clocks keep track of time at different rates, a set of “1 second” intervals 526 of the data sets 502, 504, and 506 may not necessarily capture the exact same length of time). Turning to an example illustrated in FIG. 5C, a first device 530a captures ECG data 532a at a medical scene, while a second device 530b records audio data 532b at the scene, including a set of heartbeat detect tones 536 issued by the first device 530a.

During a recording phase 534a, in some implementations, the first device 530a issues a timing synchronization message 538a to the second device 530b. The second device 532, responsive to receipt of the synchronization message 538a, records a timing event marker 540a capturing both a sent time 542a (e.g., 9:01:00) according to the first device 530a as well as an actual time 544a (e.g., 8:58:16) according to the second device 530b. Turning to FIG. 4B, for example, the timing event marker 540a may correspond to a timing event log 420 including an elapsed time 422 recording seconds that have elapsed since the beginning of treatment (e.g., powering on of the device that is capturing the timing event log 420, beginning of data capture by the device that is capturing the timing event log 420, etc.), as well as an actual time 424 recording the clock time of the device 428 capturing the timing event log 420 and a sent time 430 recording the clock time at which a synchronization message corresponding to the data row was issued by a master device in communication with the device that is capturing the timing event log 420. As illustrated, the elapsed time 422, the actual time 424, and the sent time 430 are each recorded to hundredths of a second, illustrated by way of example. The example timing event log 420 additionally includes, in some embodiments, a unique message identifier (UID) 426 corresponding to each received synchronization message.

Returning to FIG. 5C, as illustrated in the recording phase 534a, each tone 536 recorded by the second device 530b aligns in real-time with a heartbeat peak captured in the ECG data 532a. However, during a playback phase 544a, due to a difference between a length of a “1 second” interval 546a according to the clock of the first device 530a and the length of a “1 second” interval 546b according to the clock of the second device 530b, the timing of the tones 536a effectively become mapped to the longer “1 second” interval 546a of the master device 530a, thus causing misalignment between the ECG data 532a and the audio data 532b.

Turning to FIG. 5D, during a second example recording phase 534b, the first device 530a issues both the first synchronization message 538a and a second synchronization message 538b at a later time, resulting in a second timing event marker 540b. Using the information in the first timing event marker 540a and the second timing event marker 540b, a difference in clock speeds between the first device 530a and the second device 530b can be estimated, resulting in a correction which allows, in a playback phase 544b, for alignment between the tones 536a of the audio data 532b and the detected heartbeats of the ECG data 532a. For example, in analyzing a ratio of the difference in actual times 544a, 544b to the difference in sent times 542a, 544b, a difference in clock speed can be estimated. The estimation, for example, may be improved through additional samples and/or more distance samples (e.g., spread out over a longer period of time) to better capture a clock cycle adjustment using timestamps that are considered accurate, for example, to one hundredth of a second, or to more or less level of granularity.

Turning to FIG. 5E, the compression data set 502, the ECG data set 504, and the therapy data set 506, are illustrated in a third arrangement 560. In this arrangement, rather than the master device 512 capturing timing event records 552a, 522b, the master device 512 issues a series of timing communications 562a, 562b which are received by each of the second device 514 and the third device 516. As illustrated, the second device 514 receives timing messages 568a, 568b and the third device 516 receives timing messages 570a, 570b. The timing communications 562a, 562b, in some implementations, are unidirectional broadcast communications recognized by each of the second device 514 and the third device 516. In other implementations, the timing communications 562a, 562b are bi-directional communications. For example, although illustrated as 562a, the timing marker of the master device 512 may represent issuance of multiple wired and/or wireless direct communications with the other devices 514, 516. Although in the illustration the messages 568a and 570a appear to reach each of the second device 514 and the third device 516 at approximately the same time as they were issued by the master device 512, a transit delay and/or a processing delay at one or both of the devices 514, 516 may move the receipt time further into the future than is illustrated. For example, wireless transit delay may be greater than a wired connection to one of the devices 514, 516.

Responsive to the timing communications 562a, 562b, each of the second device 514 and the third device 516, in some implementations, register information in a timing event record 564a, 564b respectively. The timing event records 564a, 564b, as illustrated in the example of FIG. 5E, may collect an elapsed time, an actual time (e.g., clock time of the respective device), and a sent time (e.g., the actual time as recognized by the master device 512 when it issued each of the timing communications 562a, 562b. Although illustrated as separate timing event records 564a, 564b, in other embodiments, one or both of the second device 514 and the third device 516 may register timing events along with other device events in a device event log or data store. Further, although not illustrated, in certain embodiments, the master device 512 also collects a timing event record registering information regarding each timing event issued. For example, in the circumstance of bi-directional communications, the master device 512 may register times and device identifiers associated with responses from each device 514, 516.

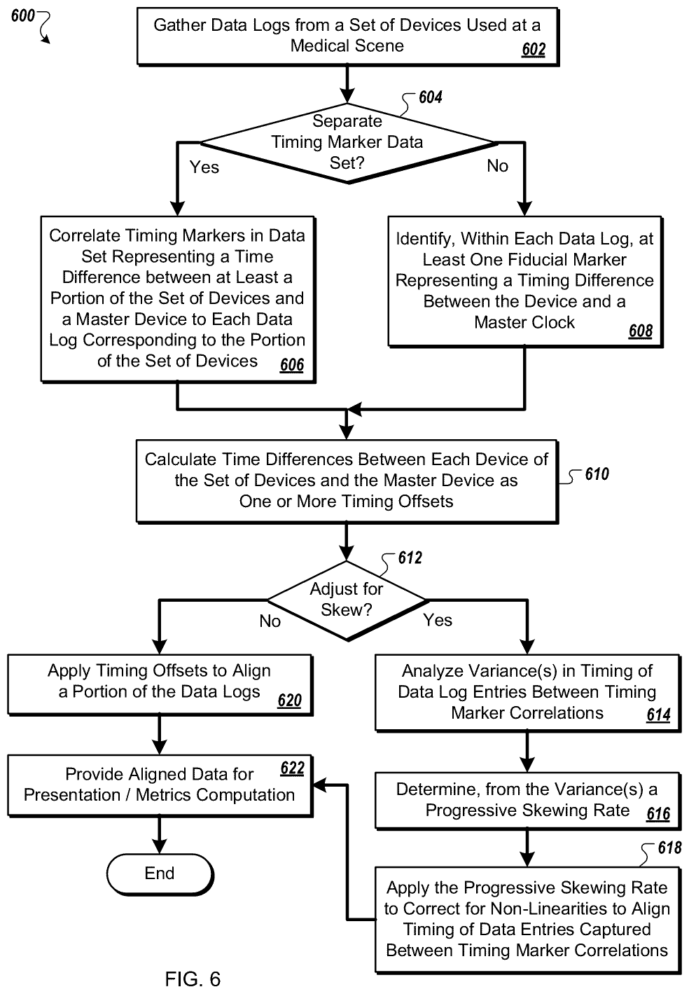

FIG. 6 is a flow chart of an example method 600 for aligning data timing of collected data sets. The method 600, for example, may be performed within the cloud computing environment 115 of FIG. 1A and FIG. 1C and/or a networked computing architecture 300 of FIG. 3. For example, portions of the method 600 may be performed by the companion device 119 of FIG. 1B, a timing alignment engine 334a of the cloud analytics platform 310, and/or a timing alignment engine 334b of the clinical or mobile application 312, the timing alignment engine 334c of one of the medical devices 302, and/or the timing alignment engine 334d of the monitoring/data device(s) 306 of FIG. 3.

In some implementations, the method 600 begins with gathering data logs from a set of devices used at a medical scene (602). The data logs may be gathered during use (e.g., in an ongoing manner) and/or after the patient has been treated. The data logs, for example, may be gathered in part along with the collection of timestamps as described in relation to operations 210 and 212 of FIG. 2A. For example, data may be collected by the monitoring/data devices 306 of FIG. 3 as part of the session data 326. As described in relation to operation 220 of FIG. 2A, at least a portion of the data logs may be uploaded to a cloud platform. For example, as illustrated in FIG. 3, the data log 326a may be uploaded to the cloud analytics platform 310 from the medical device(s) 302 via the network 308. A communications engine 338a of the cloud analytics platform 310 (e.g., an API, storage server, etc.) may enable upload to the cloud analytics platform 310. Further, a portion of the data logs may be provided in the form of sensor signals 336 provided to the cloud analytics platform 310 via the network 308 from the sensor device(s) 304, as shown in FIG. 3. The session data 326, data log 324a, and/or sensor signals 336, in a further example, may be transferred to a clinical or mobile application 312 at the scene (e.g., for storage as session data 326b). For example, a communications engine 338b (e.g., API, wireless communications engine, etc.) of the clinical or mobile application 312 may enable upload of the session data 326, data log 324a, and/or sensor signals 336. In other examples, data may be downloaded from one or more devices to a storage drive (e.g., USB drive, etc.) or computing device (e.g., laptop or tablet computer, etc.) for upload to the cloud computing platform.

In some embodiments, the gathered data is archived, for example for post-treatment review and analysis. For example, a log data archiving engine 340 of the cloud analytics platform 310 may archive the data as archive data 346 for later analysis, along with data collected at a number of medical scenes during treatment of a variety of patients. The log data archiving engine 340, in some implementations, is configured to interoperate with a log data linking engine 344 for linking data from a particular medical scene together. Similarly, in some embodiments, a data archival engine 342 of the clinical or mobile application 312 may archive the session data 326b for later analysis.

In some implementations, if a separate timing marker data set has been collected by a master timing device (604), timing markers in the data set representing a time difference between at least a portion of the set of devices and a master device are correlated to each data log corresponding to the portion of the set of devices (606). The timing alignment engine 334 of the cloud analytics platform 310, the clinical or mobile application 312, the medical device(s) 302, or the monitoring/data device(s) 306 may correlate the timing markers. If the timing markers were captured as described in relation to FIG. 2A, correlating the timing markers, for example, may include identifying one or more timing markers in the timing marker data set, by device identifier, corresponding to a data log captured by the device having the device identifier. If the timing markers were captured, as described in relation to FIG. 2B, correlating the timing markers may include matching, by device identifier, a timing marker set to its corresponding data log(s) captured by the same device identifier.

In some implementations, if timing markers are embedded within separate data logs (604), at least one fiducial marker representing a timing difference between the device and the master clock is identified within each data log (608). As discussed in relation to FIG. 2A, for example, the fiducial markers may be collected periodically such as, in some examples, every one minute, every few minutes, every five minutes, every ten minutes, or at least two to four times per hour. Where more than one fiducial marker has been collected, each fiducial marker may be identified.

In some implementations, the time differences between each device of the set of devices and the master device are calculated as timing offsets to the master clock (610). The time difference, for example, may be calculated by determining a difference between the device time and the master clock time, as logged. In some embodiments, the timing offset has already been logged as a timing marker (e.g., as a current device time plus a timing offset calculated at the time of logging the fiducial time marker). Where multiple timing markers were captured for a given device, a timing offset corresponding to each timing marker may be calculated. As shown in FIG. 4B, for example, a series of timing markers have been collected in the timing event log 420.

In some implementations, if skew adjustment is desired (612), variances in timing of data log entries between timing marker correlations are analyzed (614). The variance, for example, may demonstrate drift between the timestamps of the master clock and timestamps obtained from one or more devices. Depending upon the frequency of timing markers used for alignment, the drift may be inconsequential or noticeable. The variances, for example, may cause a non-linear offset between the master clock and the particular device clock in the time progression of data entries of the device's data log. In analyzing the variance(s) between entries, for example, it may be determined if a significant non-linearity is detected.

In some implementations, a progressive skewing rate is determined from the variances (616). The progressive skew rate, for example, may be represented by an equation using an initial offset plus a progressive adjustment based upon a time differential between each given timestamp of each data entry and the initial offset.

In some implementations, the progressing skewing rate is applied to correct for non-linearities to align timing of data entries captured between timing marker correlations (618). The progressive skewing rate, for example, may be applied to data as it is read from the data log. For example, only a portion of the data log may be adjusted, based on the end usage of the data being aligned. In another example, the timestamps in the data log may be adjusted using the progressive skewing rate. In this manner, the data may be used for different purposes without re-calculating the offset.

In some implementations where there is no adjustment for skew (612), timing offsets are applied to align the data in the data logs (620). Where multiple timing markers were captured for a given device, a timing offset corresponding to each timing marker may be applied to adjusting data captured at the time of and/or after logging of the particular timing marker.

In some implementations, whether or not skew adjustment is applied (612), the aligned data is provided for presentation and/or metrics computation (622). For example, a graphical user interface engine 340a of the cloud analytics platform 310 or a graphical user interface engine 340b of the clinical or mobile application 312 of FIG. 3 may present timing aligned graphical output and/or metrics derived from the timing-aligned data logs. In another example, an input/output (I/O) engine 342a of the medical device(s) 302 and/or an I/O engine 342b of the monitoring/data device(s) 306 may present timing aligned graphical output and/or metrics derived from the timing-aligned data logs. The graphical output, in one example, may include the graph 502 and/or graph 504 of FIG. 5A and FIG. 5B and/or the graph 532a of FIG. 5C.

Although illustrated as a particular series of operations, in some embodiments, the method 600 includes more or fewer operations. For example, where multiple timing marker data sets have been captured (604), each of the timing marker data sets may be used to correlate timing markers to data logs. The multiple timing marker data sets, in one example, may be captured by multiple devices, for example as described in relation to FIG. 2B. In another example, the multiple timing marker data sets may have been captured by a single master timing device over time (e.g., due to multiple uploads of information, as described in illustration, in relation to operations 218 and 220 of FIG. 2A). In a further example, the multiple timing marker data sets may have been captured by consecutive master timing devices, such as when a first master timing device is no longer being used for treating the patient and another master timing device takes over in providing timing requests (discussed in further detail in relation to FIG. 7A and FIG. 7B). When multiple consecutive master timing devices have been used, an offset may be calculated between a system timing of the first master device and a system timing of the second master device, and the master device offset may be applied to align timing related to data logged after the master timing device hand-off. In some embodiments, certain operations of the method 600 may be performed in a different order and/or concurrently. For example, skew adjustment for multiple devices may be calculated concurrently (e.g., operations 614 through 618). Other modifications to the method 600 are possible while remaining in the spirit and scope of the method 600.

FIG. 7A and FIG. 7B illustrate a series of medical event scenes 700, 750 beginning with an en route medical scene 700 involving a patient in an ambulance 730 and continuing to a medical facility scene 750 involving the patient once dropped off at a hospital. Between the ambulance scene 700 and the medical scene 750, multiple medical devices collecting data sets during emergency care of the patient are removed and/or replaced with a different data collection device, including an initial master timing device providing timing messages while in transit to the medical facility.

Turning to FIG. 7A, in some implementations, during treatment by ambulance personnel, a master computing device 702 (e.g., tablet computing device, computing device mounted to or integrated within the ambulance 730, etc.) issues a discovery broadcast request and one or more timing broadcast requests 710 to identify the compression device 704, the ventilation device 706, and the defibrillation device 708 and to collect timing responses 712 from each. The master computing device 702 may collect information regarding the timing responses 712 as timing data 714.

In some implementations, the compression device 704 and the ventilation device 706 are configured as peripheral devices to the defibrillation device (e.g., patient monitoring and therapy device) 708, providing in real-time or near real-time compression data 722a and ventilation data 724 for collection and analysis at the defibrillation device 708.

In some implementations, the master computing device 702 provides access to the PCR network 718, such that the defibrillation device 708 may upload peripheral log data 728a including at least portions of the compression data 722a and the ventilation data 724 and operational data 730a regarding operation of the defibrillation device 708. The defibrillation device 708 may also upload clinical metrics 726a derived from the compression data 722a, ventilation data 724, and/or operational data 730a. The clinical metrics 726a, peripheral log data 728a, and operational data 730a, for example, may be stored as log data 732a by the defibrillation device 708 and/or the master computing device 702 at least until opportunity for upload to the PCR network 718 becomes available.

In some implementations, the master computing device 702 uploads the timing data 714 and the log data 732a as timing and log data 716 to the cloud analytics platform 720 via the PCR network 718. The upload, for example, may be performed ongoing (as network availability exists), periodically, and/or upon an upload trigger event (e.g., arrival at a hospital, upload command from a medical caregiver, etc.).

Turning to FIG. 7B, in some implementations, a hospital medical scene 750 includes a portion of the devices used in the medical transit scene 700, including the compression device 704 and the defibrillation device 708. However, the manual bag mask style ventilation device 706 has been replaced with an automated ventilation device 754, and a pulse oximetry sensor 752 has been introduced.

Since the master computing device 702 remained with the ambulance 730, in some implementations, the defibrillation device 708 proceeds with issuing a discovery broadcast request followed by periodic timing broadcast requests 756. Upon recognition of loss of the master computing device 702 (e.g., based on lack of receipt of periodic timing request broadcasts), for example, the defibrillation device 708, also installed with master timing collection capabilities, may take over by issuing the timing request broadcast 756. If multiple devices are equipped with master timing collection capabilities, in some embodiments, a hierarchical selection or other negotiation may ensue to select a new master timing device. For example, since the compression device 704 is peripheral to the defibrillation device 708, the defibrillation device 708 may hierarchically take over as the master timing device.

In some implementations, the defibrillation device 708 collects timing responses 758 from the compression device 704, the ventilation device 754, and the pulse oximetry device 752 and arranges the information as timing data 760 which may be stored for later transmission to the cloud analytics platform 720 or uploaded in real-time. The format of the timing data 760 may be the same as the timing data 714 of FIG. 7A or it may be different.

In some implementations, at the cloud analytics platform 720, the timing offset between the defibrillation device 708 and the master timing device 702 may be applied to the timing data 760 captured by the defibrillation device 708 to align all timing data 714, 760 to the same master clock (e.g., the time of the master device 702).

As with FIG. 7A, further compression data 722b may be collected by the defibrillation device 708 as well as ventilation data 762 generated by the ventilation device 754. The collected data (peripheral log data 728b), additional clinical metrics 726b, and additional operational data 730b may be stored as log data 732b and transferred (in real-time, periodically, or post treatment) to the cloud analytics platform 720 by the defibrillation device 708.

In some implementations, pulse oximetry data 764 from the pulse oximetry device 752 is uploaded separately to the cloud analytics platform 720 (e.g., in real-time, periodically, or post treatment).

FIG. 8 is a block diagram of an example system 800 including various computing devices configured for use at an emergency medical scene. The devices include a therapy monitoring device 804, one or more therapeutic medical devices 802, and rescuer feedback devices 806 for providing feedback to a team of rescuers 864. The therapeutic medical devices 802 may interface with a patient 862 via a set of patient interface elements 830. The devices 802, 804, and 806 of the system 800, for example, may be deployed at an emergency medical scene, such as the scene 100 of FIG. 1A, the scene 150 of FIG. 1B, and/or the scene 170 of FIG. 1C. The devices 802, 804, and/or 806 may represent various devices deployed at the emergency medical scenes 100, 150, and/or 170.

For example, the medical therapy may be electrical therapy (e.g., defibrillation, cardiac pacing, synchronized cardioversion, diaphragmatic or phrenic nerve stimulation), and the medical device 802 may be a defibrillator, a defibrillator/monitor, a mechanical ventilator such as the Z Vent Ventilator provided by ZOLL Medical Corporation, and/or another medical device configured to provide electrotherapy. As another example, the medical therapy may be chest compression therapy for treatment of cardiac arrest and the medical device 802 may be a mechanical chest compression device such as a belt-based chest compression device or a piston-based chest compression device. In other examples, the medical therapy may be ventilation therapy, therapeutic cooling or other temperature management, invasive hemodynamic support therapy (e.g., Extracorporeal Membrane Oxygenation (ECMO)), etc. and the medical device 802 may be a device configured to provide a respective therapy. Further, in some embodiments, the medical device 802 includes a combination of one or more of these examples. The therapeutic medical device 802 may include patient monitoring capabilities via one or more sensors 832b. In example, the medical device 802 may be the defibrillation/monitor device 108 of FIG. 1A through FIG. 1C, the medical device(s) 302 of FIG. 3, the compression device 704 of FIG. 7A and FIG. 7B, the defibrillation device 708 of FIG. 7A and FIG. 7B, and/or the ventilation device 754 of FIG. 7B.

Turning to the therapeutic medical devices 802, each medical device 802 may include a processor 808, a memory 810, a communications interface 816, one or more input devices 814, one or more output devices 812, and a therapy delivery control module 818, all interconnected by a communications bus 817. The therapy delivery control module 818 enables delivering of a medical therapy.

In some embodiments, the therapeutic medical device 802 is an integrated therapy delivery/monitoring device within a single housing. The single housing may surround, at least in part, at least a portion of the patient interface elements 830, including, for example, some therapy delivery components 832a and monitoring components such as sensors 832b. In other embodiments, the medical device 802 is a modular therapy delivery/monitoring device, with patient therapy components 832a in one unit communicatively coupled to a patient monitoring unit with monitoring components (e.g., sensors 832b) but without therapy delivery components 832a.

The patient interface elements(s) 830 include one or more therapy delivery component(s) 832a and/or one or more sensor device(s) 832b. The therapy delivery component(s) 832a are configured to deliver therapy to the patient 862 and may be configured to couple to the patient 862. For example, the therapy delivery component(s) 832a may include one or more of electrotherapy electrodes including defibrillation electrodes and/or pacing electrodes, chest compression devices (e.g., one or more belts or a piston), ventilation devices (e.g., a mask and/or tubes), drug delivery devices, etc. The medical device 802 may include the one or more therapy delivery component (s) 832a and/or may be configured to couple to the one or more therapy delivery component(s) 832a in order to provide medical therapy to the patient 862. The therapy delivery component(s) 832a may be configured to couple to the patient 862. For example, one of the rescuers 864 may attach the electrodes to the patient 862 and the medical device 802 (e.g., a defibrillator or defibrillator/patient monitor) may provide electrotherapy to the patient 862 via the defibrillation electrodes. In some embodiments, the medical device 802 couples to at least a portion of the patient interface elements(s) 830 via a wired or wireless communications protocol supported by the communications interface 816.

The medical device(s) 802, in some implementations, include, incorporate, and/or are configured to couple to the one or more sensor(s) 832b. The sensor(s) 832b may be configured to provide signals indicative of sensor data to the medical device 802. The sensor(s) 832b may be configured to couple to the patient 862. In some embodiments, the medical device 802 couples to at least a portion of the sensors 832b via a wired or wireless communications protocol supported by the communications interface 816.

In some embodiments, the sensor(s) 832b include sensing electrodes 838 such as one or more cardiac sensing electrodes, a chest compression sensor 844, and/or ventilation sensors 840. The sensing electrodes 838 may be conductive and/or capacitive electrodes configured to measure changes in a patient's electrophysiology to measure the patient's ECG information. The sensing electrodes 838 may further measure the transthoracic impedance and/or a heart rate of the patient 862. The one or more sensors 832b may generate signals indicative of physiological parameters of the patient 862. For example, the physiological parameters may include one or more of at least one vital sign, an ECG, blood pressure, heart rate, pulse oxygen level, respiration rate, heart sounds, lung sounds, respiration sounds, tidal CO2, saturation of muscle oxygen (SMO2), arterial oxygen saturation (SpO2), cerebral blood flow, electroencephalogram (EEG) signals, brain oxygen level, tissue pH, tissue fluid levels, physical parameters as determined via ultrasound images, parameters determined via near-infrared reflectance spectroscopy, pneumography, and/or cardiography, etc. The ultrasound images may include ultrasound images of a patient's heart, carotid artery, and/or other components of the cardiovascular system. Additionally or alternatively, the one or more sensors 832b may generate signals indicative of chest compression parameters, ventilation parameters, drug delivery parameters, fluid delivery parameters, etc.

In some embodiments, the sensor(s) 832b include ventilation sensors 840. The ventilation sensor(s) 840 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, and combinations thereof.

In some embodiments, the sensor(s) 832b include temperature sensors 842. The temperature sensors 842 may include an infrared thermometer, a contact thermometer, a remote thermometer, a liquid crystal thermometer, a thermocouple, a thermistor, etc. and may measure patient temperature internally and/or externally.

In some embodiments, the sensor(s) 832b include chest compression sensor(s) 844. The chest compression sensor(s) 844 may include one or more motion sensors including, for example, one or more accelerometers, one or more force sensors, one or more magnetic sensors, one or more velocity sensors, one or more displacement sensors, etc. The chest compression sensor(s) 844 may be incorporated into, in some examples, a compression puck, a smartphone, a hand-held device, a wearable device, etc. The chest compression sensors 844 may be configured to detect chest motion imparted by one of the rescuers 864 and/or an automated chest compression device (e.g., a belt system, a piston system, etc.). The chest compression sensors 844 may provide signals indicative of chest compression data including displacement data, velocity data, release velocity data, acceleration data, compression rate data, dwell time data, hold time data, blood flow data, blood pressure data, etc. In some implementations, the sensing electrodes 838 and/or the electrotherapy electrodes 834a may include or be configured to couple to the chest compression sensors 844.

In addition to delivering therapy to the patient 862, in some implementations, the therapy delivery component(s) 832a may include, be coupled to, and/or function as sensors and provide signals indicative of sensor data to the medical device 802. For example, the defibrillation electrodes may be configured as cardiac sensing electrodes 838 as well as electrotherapy electrodes 834a and may provide signals indicative of transthoracic impedance, electrocardiogram (ECG), heart rate and/or other physiological parameters. As another example, a therapeutic cooling device may be an intravenous cooling device. Such a cooling device may include an intravenous (IV) device 834c as a therapy delivery component 832a configured to deliver cooling therapy and sense the patient's temperature as a temperature sensor 842. For example, the IV device may be a catheter that includes saline balloons configured to adjust the patient's temperature via circulation of temperature controlled saline solution. In addition, the catheter may include a temperature probe configured to sense the patient's temperature. As a further example, an IV device 834c may provide therapy via drug delivery and/or fluid management. The IV device 834c may also monitor and/or enabling monitoring of a patient via blood sampling and/or venous pressure monitoring (e.g., central venous pressure (CVP) monitoring).

The medical device 802, in some implementations, is configured to receive sensor signals from the therapy delivery component(s) 832a and/or the sensor(s) 832b and to process the sensor signals to determine and collect patient data. The patient data may include patient data which may characterize a status and/or condition of the patient 862 (e.g., physiological data such as ECG, heart rate, respiration rate, temperature, pulse oximetry, non-invasive hemoglobin parameters, capnography, oxygen saturation (SpO2), end tidal carbon dioxide (EtCO2), invasive blood pressure (IBP), non-invasive blood pressures (NIBP), tissue pH, tissue oxygenation, Near Infrared Spectroscopy (NIRS) measurements, etc.). Additionally or alternatively, the patient data may characterize the delivery of therapy (e.g., chest compression data such as compression depth, compression rate, etc.) and/or the patient data may characterize a status and/or condition of the medical equipment used to treat the patient (e.g., device data such as shock time, shock duration, attachment of electrodes, power-on, etc.). The patient data may be collected by the processor 808 via the communications interface 816 and stored to the memory 810.

In some implementations, the therapy delivery control module 818 is configured to couple to and control the therapy delivery component(s) 832a. In one example, the therapy delivery control module 818 of the medical device 802 may be an electrotherapy delivery circuit that includes one or more capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 818 may be a compression device electro-mechanical controller configured to control a mechanical compression device. As a further example, the therapy delivery control module 818 may be an electro-mechanical controller configured to control drug delivery, temperature management, ventilation, and/or other type of therapy delivery.

A user, in some implementations, initiates therapy and/or adjusts therapy parameters via one or more input devices 814 of the medical device 802. The input devices 814, in some examples, may include one or more virtual or physical controls, such as buttons, switches, dials, and/or touch controls presented at a touch screen display of the medical device 802. Further, the input devices 814 may include a communications interface 816 for receiving instructions from a remotely located control device and/or remote application such as a smart phone application.

In some implementations, a user receives feedback from the medical device 802 and/or reviews a portion of the patient data via one or more output devices 812. The output devices 812, in some examples, may include a display screen for presenting a portion of the patient data and/or metrics derived therefrom (e.g., as calculated by the processor 808). The output devices 812 may include a speaker for generating alarms, audible instructions, or pacing tones (e.g., for prompting timing of chest compressions, ventilations, etc.).

Although shown as separate elements, one or more of the elements of the medical device 802 may be combined into one or more discrete components and/or may be part of the processor 808. The processor 808 and the memory 810 may include and/or be coupled to associated circuitry to perform the functions described herein.

In some implementations, the medical device 802 is configured to communicate with the therapy monitoring device 804 and/or the set of rescuer feedback devices 806 via the communications interface 816. The communications interface 816 may be configured to communicate with the therapy monitoring device 804 via wired and/or wireless communicative couplings. Further, the communications interface 816 may be configured to communicate with the rescuer feedback devices 806 via wireless communicative couplings. The communications interface 816 may be configured to communicate using one or more communications protocols. In some examples, the communications interface 816 may be configured to communicate via a wireless coupling such as a cellular network including FirstNet by First Responder Network Authority, EDGE, 3G, 4G, and 5G wireless cellular systems. The wireless network can also include Wi-Fi®, Bluetooth®, Zigbee®, or another wireless form of communication.

In some implementations, the medical device 802 receives activity values from the rescuer feedback devices 806.

In some implementations, the medical device 802 provides the patient data to the therapy monitoring device 804 over a communications coupling 858a. For example, a communications interface 828 of the therapy monitoring device 804 may establish with communications coupling 858a with the communications interface 816 of the medical device 802. The processor 808 of the medical device 802 may generate the patient data and provide the patient data to the therapy monitoring device 804, via the communications interface 816, in real-time or near real-time to support oversight of the therapy at the therapy monitoring device 804. In some embodiments, a portion of patient data stored to the therapy monitoring device 804 is generated by the therapy monitoring device 804. The therapy monitoring device 804, for example, may include one or more sensors 823 configured to collect additional patient data.

In some implementations, the therapy monitoring device 804 stores the patient data to a memory 822. A processor 820 of the therapy monitoring device 804 may generate one or more metrics from the patient data and/or apply one or more rules to the patient data for identifying problems with the therapy (e.g., delivery problem, equipment problem, etc.). The processor 820 may present the patient data, metrics, and/or alarms related to problems via one or more output devices 824. For example, the therapy monitoring device 804 may display the compression data 502, the ECG data 504, and/or the therapy event data 506 of FIG. 5A and FIG. 5B, and/or the ECG data 532a of FIG. 5C. A user may interact with the therapy monitoring device 804 to review various aspects of the patient data using one or more user input devices 826. The user input devices, in some examples, can include buttons, a keyboard, a touch screen, a voice recognition microphone, or a gesture recognition sensor.

The therapy monitoring device 804, in some embodiments, is a portable computing device such as, in some examples, a laptop computer, tablet computer, mobile phone, or other handheld computing device. The therapy monitoring device 804, in particular examples, may be the supervisor device 119 of FIG. 1B or the computing device 702 of FIG. 7A.

Turning to the rescuer feedback devices 806, in some implementations, a personal feedback device is allocated to each rescuer 864 at an emergency scene. The rescuer feedback devices 806, for example, may include the wearable device 111 of FIG. 1A through FIG. 1C. As illustrated, each feedback device may include a processor 846, a memory 848, one or more sensors 850, one or more output devices 852, one or more input devices 854, and at least one communications interface 856 for communicating with the therapy monitoring device 804 and/or the therapeutic medical device(s) 802.

The communications interface 856, for example, may receive feedback data from the therapy monitoring device 804 and/or the therapeutic medical device(s) 802, and the processor 846 may prepare the information for presentation via the output device(s) 852, such as a display interface.

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, the method 200 of FIG. 2A, the method 250 of FIG. 2B, and/or the method 600 of FIG. 6.

These computer program instructions can direct a computing device or other programmable data processing apparatus to function in a particular manner, such that the instructions stored in the computer-readable medium produce an article of manufacture including instruction means which implement the function/operation specified in the illustrated process flows.

Embodiments of the present description rely on network communications. As can be appreciated, the network can be a public network, such as the Internet, or a private network such as a local area network (LAN) or wide area network (WAN) network, or any combination thereof and can also include PSTN or ISDN sub-networks. The network can also be wired, such as an Ethernet network, and/or can be wireless such as a cellular network including EDGE, 3G, 4G, and 5G wireless cellular systems. The wireless network can also include Wi-Fi®, Bluetooth®, Zigbee®, or another wireless form of communication. The network, for example, may support communications between the defibrillation device 108 and the sensors 122, 124, and 126 and/or the devices 111, 113 of FIG. 1A, between the defibrillation device 108 and the cloud computing environment 115 of FIG. 1A, the defibrillation device 108 and the tablet computing device 119 and/or the tablet computing device 152 and the tablet computing device 119 of FIG. 1B, the sensors 122, 124, 126 and/or devices 111, 113 and the cloud computing environment 115 of FIG. 1C, the medical device(s) 302, sensor device(s) 304, and/or monitoring/data device(s) 306 and the cloud analytics platform 310 and/or clinical or mobile application 312 of FIG. 3, the compression device 704, ventilation device 706, and/or defibrillation device 708 of FIG. 7A and the computing device 702, the compression device 704 and/or the ventilation device 706 and the defibrillation device 708 of FIG. 7A, the computing device 702 and the cloud analytics platform 720 of FIG. 7A, the defibrillation device 708 and the cloud analytics platform 720 of FIG. 7B, the pulse oximetry device 752 and the cloud analytics platform 720 of FIG. 7B, and/or the ventilation device 754 and the defibrillation device 708 of FIG. 7B.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display. The display controller and display may enable presentation of the data graphics illustrated, in some examples, in FIG. 5A and FIG. 5B, FIG. 5C, and/or FIG. 5D.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

The functions and features described herein may also be executed by various distributed components of a system. For example, one or more processors may execute these system functions, where the processors are distributed across multiple components communicating in a network. The distributed components may include one or more client and server machines, which may share processing, in addition to various human interface and communication devices (e.g., display monitors, smart phones, tablets, personal digital assistants (PDAs)). The network may be a private network, such as a LAN or WAN, or may be a public network, such as the Internet. Input to the system, in some examples, may be received via direct user input and/or received remotely either in real-time or as a batch process.

Although provided for context, in other implementations, methods and logic flows described herein may be performed on modules or hardware not identical to those described. Accordingly, other implementations are within the scope that may be claimed.

In some implementations, a cloud computing environment, such as Google Cloud Platform™ or Amazon™ Web Services (AWS™), may be used perform at least portions of methods or algorithms detailed above. The processes associated with the methods described herein can be executed on a computation processor of a data center. The data center, for example, can also include an application processor that can be used as the interface with the systems described herein to receive data and output corresponding information. The cloud computing environment may also include one or more databases or other data storage, such as cloud storage and a query database. In some implementations, the cloud storage database, such as the Google™ Cloud Storage or Amazon™ Elastic File System (EFS™), may store processed and unprocessed data supplied by systems described herein. For example, the contents of the data logs 116 of FIG. 1A through FIG. 1C, the contents of the data logs 324, the session data 326b, and/or the archive data 346 of FIG. 3, the log data 732a of FIG. 7A, and/or the log data 732b of FIG. 7B may be maintained in a database structure.

The systems described herein may communicate with the cloud computing environment through a secure gateway. In some implementations, the secure gateway includes a database querying interface, such as the Google BigQuery™ platform or Amazon RDS™. The data querying interface, for example, may support access by the cloud analytics platform 310 and/or the clinical or mobile application 312 of FIG. 3.

In some embodiments, an edge server is used to transfer data between sensors, medical devices, and/or patient monitoring devices and a cloud-based analytics environment according to various embodiments described herein. The edge server, for example, may be a computing device configured to execute processor intensive operations that are sometimes involved when executing machine learning processes, such as natural language processing (NLP) operations. An edge server may include, for example, one or more GPUs that are capable of efficiently executing matrix operations as well as substantial cache or other high-speed memory to service the GPUs. An edge server may be a separate, ruggedized physical device that travels with EMS personnel in the field. An edge server may be incorporated into other EMS field equipment such as a medical device or patient monitoring device. Alternatively or additionally, an edge server may be located within a carrying case for a medical device. An edge server, in a further example, may be incorporated into the communications and processing capabilities of an EMS vehicle or otherwise be located within the EMS vehicle.

In some implementations, the defibrillator 108 of FIG. 1A, the tablet 119 of FIG. 1B the clinical or mobile application 312 of FIG. 3, and/or the computing device 702 of FIG. 7A operate as the edge server, for example if the processing capability of these devices is sufficient to provide computing services associated with an edge server. As illustrated in FIG. 1B, for example, any of the defibrillator 108, the tablet 119, and the tablet 152 may be designed to perform as the edge server, while the other of these devices may all be considered as local devices to the edge server, as well as the various sensor devices at the scene 150, because the devices 108, 119, and 152 are located in proximity to one another and to the EMS personnel 158 as well as the patient 102. The server environment 115 of FIG. 1A, conversely, may be or include a remote device because the server environment 115 may be hosted in a cloud service including one or more cloud servers located remotely from all of the devices at the scene 100. The edge server, in the example scene 100, can be used to move more computing capability into the local environment so that any computation intensive data processing and/or analytics can run accurately and efficiently. Further, as illustrated in the scene 150 of FIG. 1B, an edge server may be incorporated into a device or added as another device at the scene 150 to support the tablet computer 119 in the absence of a connection with a remote cloud server.

Regardless of its physical form, an edge server can be configured to interoperate with other devices proximate or local to the edge server, directly or via a network. For instance, the edge server can include a wireless network interface (e.g., a PAN interface, LAN interface, WAN interface, or the like) through which the edge server can communicate with the other devices at the scene and/or a server (e.g., cloud) environment.

In a reciprocal manner, the devices at the scene, in some embodiments, are configured to connect directly or indirectly to and interoperate with the edge server via a short-range wireless connection, such as a PAN connection or a LAN connection. In an illustrative example, the defibrillator 108 and/or the tablet 119 of FIG. 1B may communicate via a short range wireless connection to an edge server and, in turn, the edge server may communicate via a long range wireless connection to a server environment.

In some implementations, the method 200 of FIG. 2A, the method 250 of FIG. 2B, and/or the method 600 of FIG. 6 may be configured to be performed in part by an edge server or a device interoperating with an edge server. The device interoperating with the edge server, for example, may share processing functionality with the edge server via one or more APIs implemented by the processes.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A system for aligning data timing of a plurality of data sets collected by a plurality of devices used at a medical scene, the system comprising:
a first device of the plurality of devices comprising
a non-volatile data store,
at least one wireless communications interface configured for bi-directional communications using a wireless communication protocol, and
processing circuitry configured to
establish, using the at least one wireless communications interface, wireless communication with at least a portion of the plurality of devices, and
repeatedly, for each respective device of the at least the portion of the plurality of devices during treatment of a patient at the medical scene,
receive, via the at least one wireless communications interface, a message including a current device time as determined by the respective device,
determine a receipt time of receipt of the message, and
store, to the non-volatile data store, a timing correlation based on the current device time, the receipt time, and a device identifier of the respective device,
wherein each respective timing correlation for the at least the portion of the plurality of devices is configured to be applied as an offset to timestamps of a corresponding data set of the plurality of data sets, each data set collected by a respective device of the plurality of devices, to align timing of the plurality of data sets, thereby enabling combined analysis across at least a portion of the plurality of data sets.

2. (canceled)

3. The system of claim 1, wherein establishing the wireless communication comprises broadcasting a timing request to at least a portion of the plurality of devices.

4. The system of claim 1, wherein establishing the wireless communication comprises broadcasting a data request to at least a portion of the plurality of devices.

5.-6. (canceled)

7. The system of claim 1, wherein the current device time is provided at least to a tenth of a second accuracy.

8. The system of claim 1, wherein, for one or more devices of the plurality of devices, the message including the current device time comprises at least one data value collected from the respective device.

9. The system of claim 1, wherein at least one device of the at least the portion of the plurality of devices is configured to transmit, in real time, the current device time and at least one data value collected from the respective device.

10. The system of claim 9, wherein the at least one device comprises a sensor device, and the at least one data value is a sensed value.

11. The system of claim 1, wherein:
the processing circuitry is further configured to periodically issue a request to the at least the portion of the plurality of devices; and
receiving the message comprises, for one or more devices of the at least the portion of the plurality of devices, receiving a response from the request.

12. The system of claim 11, wherein periodically issuing the request comprises broadcasting the request wirelessly to all devices within range.

13. The system of claim 11, wherein periodically issuing the request comprises issuing the request once every one to five minutes.

14. The system of claim 1, wherein:
at least one device of the at least the portion of the plurality devices is configured to transmit, in real time, a data stream comprising a plurality of data values; and
the processing circuitry is further configured to store, to the non-volatile data store, a data log for the at least one device.

15. The system of claim 14, wherein storing the timing correlation comprises storing, in the data log, the timing correlation.

16. The system of claim 14, wherein each device of the at least one device is a medical device or a sensor device, and the first device is a patient monitoring device.

17. The system of claim 14, wherein the data stream comprises data collected by two or more devices connected to the at least one device as two or more peripheral devices.

18.-19. (canceled)

20. The system of claim 14, wherein:
the at least one device comprises two or more devices; and
the processing circuitry is further configured to, for each respective device of the at least one device, apply the offset derived from the respective timing correlation to at least a portion of a plurality of timestamps of a respective data log of the respective device.

21. The system of claim 1, wherein:
two or more devices of the at least the portion of the plurality devices are configured to transmit, in real time, a respective data stream comprising a respective plurality of data values; and
the processing circuitry is further configured to apply, in real-time, timing correlations corresponding to each respective device of the two or more devices to the respective data stream of the respective device to synchronize the respective plurality of data values of the respective data streams of the two or more devices.

22. The system of claim 21, wherein the processing circuitry is further configured to present, at a display in real-time or near real-time, co-registered graphical outputs of the respective plurality of data values corresponding to the respective data streams of at least two devices of the two or more devices.

23. (canceled)

24. The system of claim 21, wherein the processing circuitry is further configured to present, at a display in real-time or near real-time, one or more time-based metrics combining synchronized data values of the respective data streams of at least two devices of the two or more devices.

25.-28. (canceled)

29. The system of claim 1, wherein storing the timing correlation comprises storing the current device time, the receipt time, and the device identifier to a timing marker data file comprising correlations for each device of the at least the portion of the plurality of devices.

30. The system of claim 29, wherein the processing circuitry is further configured to provide, via a network, the timing marker data file to another computing device.

31. The system of claim 30, wherein:
the plurality of devices comprises the another computing device; and
the processing circuitry is configured to negotiate, with the another computing device, for role as master device updating the timing marker data file.

32. The system of claim 1, wherein the processing circuitry is configured to upload a set of timing correlations corresponding to the at least the portion of the plurality of devices via a network to a cloud-based data analytics platform, the cloud-based data analytics platform comprising processing logic configured to:
receive, separately, at least a subset of the plurality of data sets collected by the plurality of devices; and
apply the set of timing correlations to the at least the portion of the plurality of data sets corresponding to the at least the portion of the plurality of devices to synchronize data values of the at least the portion of the plurality of data sets.

33.-60. (canceled)
